(19) United States
(12) Patent Application Publication
(10) Pub. No.: US 2016/0168132 A1
(43) Pub. Date: Jun. 16, 2016

(54) DI(HETERO)ARYLAMIDES AND SULFONAMIDES, METHODS FOR THEIR PREPARATION AND THERAPEUTIC USES THEREOF

(71) Applicant: MINORYX THERAPEUTICS S.L., Mataró - Barcelona (ES)

(72) Inventors: Xavier BARRIL ALONSO, Mataró, Barcelona (ES); Ana Maria GARCÍA COLLAZO, Mataró, Barcelona (ES); Juan AYMAMI BOFARULL, Mataró, Barcelona (ES); Marc REVÉS VILAPLANA, Barcelona (ES); Rodolfo LAVILLA GRIFOLS, Castelldefels, Barcelona (ES); Marc MARTINELL PEDEMONTE, Mataró, Barcelona (ES)

(73) Assignee: Minoryx Therapeutics S.L., Mataró - Barcelona (ES)

(21) Appl. No.: 14/908,944

(22) PCT Filed: Jul. 30, 2014
(86) PCT No.: PCT/EP2014/066392

§ 371 Date: Jan. 29, 2016

(30) Foreign Application Priority Data
Jul. 31, 2013 (EP) 13382314.6

(51) Int. Cl. C07D 405/12; C07D 239/48; C07C 235/56; C07D 213/74; C07C 255/60; C07D 213/81; C07D 213/82; C07D 401/12; C07D 215/20; C07C 233/80; C07C 311/21

(52) U.S. Cl. CPC C07D 405/12; C07C 233/80; C07D 239/48; C07C 235/56; C07D 213/74; C07C 311/21; C07D 213/81; C07D 213/82; C07D 401/12; C07D 215/20; C07C 255/60

(57) ABSTRACT

The present invention refers to compounds of formula (I): as well as to a method for their preparation, pharmaceutical compositions comprising the same, and use thereof for the treatment and/or prevention of conditions associated with the alteration of the activity of β-galactosidase, specially galactosidase beta-1 or GLB1, including GM1 gangliosidoses and Morquio syndrome, type B.

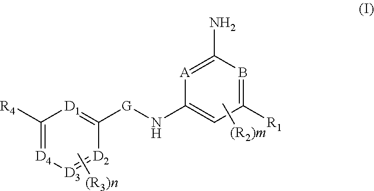

### FIELD OF THE INVENTION

The present invention is related to di(hetero)arylamides and sulfonamides, with new processes for their preparation and to the use thereof for the treatment and/or prevention of conditions associated with the alteration of the activity of beta galactosidase, specially galactosidase beta-1 or GLB1, including GM1 gangliosidoses and Morquio syndrome, type B.

### BACKGROUND OF THE INVENTION

GM1 gangliosidosis and Morquio B syndrome, both arising from beta-galactosidase (GLB1) deficiency, are very rare lysosomal storage diseases with an incidence of about 1:100,000-1:200,000 live births worldwide (Caciotti A. et al Biochim Biophys Acta 2011 July; 1812(7) 782-890). Said conditions associated with GLB1 are known to be caused by a deficiency of the enzyme 1-galactosidase due to mutations in the GLB1 gene.

β-galactosidase cleaves β-galactoses from different substrates, and deficiencies in its activity cause said substrates (i.e. gangliosides, and oligosaccharides carrying terminal β-linked galactose, such as ganglioside GM-1 and glycosaminoglycans such as keratin sulfate) to accumulate in patients suffering from conditions associated with GLB1 activity such as GM1 gangliosidosis and Morquio B syndrome.

Suzuki et al. Cell. Mol. Life Sci. 65 (2008) 351-353 reported that the mutations of the GLB1 gene result in an unstable mutant β-galactosidase enzyme protein with normal or near-normal biological activity. The mutant enzyme protein seems to be unstable at neutral pH in the endoplasmic reticulum (ER)/Golgi apparatus, and rapidly degraded because of inappropriate molecular folding and this is the reason its impaired activity. The authors also reported that the use of a competitive inhibitor binding to misfolded mutant protein as a molecular chaperone (i.e. a small molecule that interacts with a misfolded protein to achieve a recovery on its activity) resulted in the formation of a stable molecular complex at neutral pH. The protein-chaperone complex was safely transported to the lysosome, where it dissociates under the acidic conditions. In this way the mutant enzyme remained stabilized, and its catalytic function was enhanced.

Several patents and publications have since then explored the use of chaperones to treat conditions associated with the alteration of the activity of GLB1: WO 2008/034575 A, WO 2006/100586 A, WO 2009/049421 A, WO 2010/046517, EP 1 433 776 A and Ogawa S. et al. Bioorg. Med. Chem. 10(6), 1967-1972 (2002).

Therefore, small molecules capable of binding alosterically to mutated 3-galactosidase enzyme thereby stabilizing the enzyme against degradation (chaperones) constitute an important therapeutic target in conditions associated with the alteration of the activity of beta galactosidase, specially galactosidase beta-1 or GLB1.

The inventors have now surprisingly found that compound of general formula (I) are capable of binding to beta galactosidase thereby stabilizing the enzyme against denaturation.

### SUMMARY OF THE INVENTION

A first aspect of the invention (which is also designated as embodiment 1) is a compound of general formula (I),

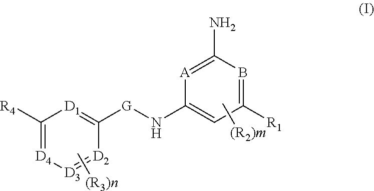

wherein:
A or B are independently selected from nitrogen and —CH═;
D₁, D₂, D₃ and D₄ are nitrogen or ═CH— with the proviso that 0, 1 or 2 of D₁, D₂, D₃ and D₄ are nitrogen while the rest are —CH═;
G is selected from —C(═O)— and —SO₂—;
R₄ is selected from halogen, methyl, —CF₃ and —OCF₃;
R₁ is selected from hydrogen, halogen, hydroxy, —CN, —ORa, —SRa, —N(Rb)₂, —C₁₋₄ alkyl, —C(═O)ORc, —C(═O)N(Rb)₂, —SO₂N(Rb)₂, —C₃₋₆ cycloalkyl and —C₃₋₇ heterocyclyl; said alkyl, cycloalkyl and heterocyclyl groups are optionally substituted with 1, 2 or 3 groups independently selected from halogen, hydroxy, —C₁₋₄ alkyl, —N(Rb)₂, methoxy, -haloC₁₋₄alkyl, -dihaloC₁₋₄alkyl, -trihaloC₁₋₄alkyl, halomethoxy, dihalomethoxy, and trihalomethoxy;
each Ra or Rb independently represents, hydrogen, —C(═O)Rd, —SO₂Rd, —C₁₋₄ alkyl, —C₃₋₆ cycloalkyl, —C₅₋₁₀ aryl, —C₅₋₁₀ heteroaryl, or —C₃₋₇ heterocyclyl; said alkyl, cycloalkyl, aryl, heteroaryl or heterocyclyl groups optionally being substituted with 1, 2 or 3 groups independently selected from halogen, hydroxy, amine, —C₁₋₄ alkyl, —CN, methoxy, aryl, substituted heteroaryl, -haloC₁₋₄alkyl, -dihaloC₁₋₄alkyl, -trihaloC₁₋₄alkyl, halomethoxy, dihalomethoxy and trihalomethoxy;
each Rd or Rc independently represent hydrogen, —C₁₋₄ alkyl, —C₃₋₆ cycloalkyl, —C₅₋₁₀ aryl, —C₅₋₁₀ heteroaryl, or —C₃₋₇ heterocyclyl; said alkyl, cycloalkyl, aryl, heteroaryl or heterocyclyl groups optionally being substituted with 1, 2 or 3 groups independently selected from halogen, hydroxy, methoxy, halomethoxy, dihalomethoxy, and trihalomethoxy;
each R₃ is independently selected from hydrogen, halogen, hydroxy, —CN, —ORa′, —SRa′, —N(Rb′)₂ and —C₁₋₄ alkyl; said —C₁₋₄ alkyl group optionally being substituted with 1, 2 or 3 groups independently selected from halogen, hydroxy and —N(Rb)₂;
each Ra′ or Rb′ independently represent, on each occasion when used herein, hydrogen, —C(═O)Rd, —SO₂Rd, —C₁₋₄ alkyl, —C₃₋₆ cycloalkyl, —C₅₋₁₀ aryl, —C₅₋₁₀ heteroaryl, or —C₃₋₇ heterocyclyl; said alkyl, cycloalkyl, aryl, heteroaryl or heterocyclyl groups optionally being substituted with 1, 2 or 3 groups independently selected from halogen, hydroxy, amine, methoxy, substituted aryl, substituted heteroaryl, halomethoxy, dihalomethoxy and trihalomethoxy;
each R₂ is independently selected from hydrogen, halogen, hydroxy, —CN, —ORa, —SRa, —N(Rb)₂, —C₁₋₄ alkyl, —C₃₋₆ cycloalkyl, —C₅₋₁₀ aryl and —C₃₋₇ heterocyclyl; said alkyl, cycloalkyl, aryl or heterocyclyl groups optionally being substituted with 1, 2 or 3 groups independently selected from halogen, hydroxy, —C₁₋₄ alkyl, —N(Rb)₂, methoxy, -haloC₁₋₄alkyl, -dihaloC₁₋₄alkyl, -trihaloC₁₋₄alkyl, halomethoxy, dihalomethoxy, and trihalomethoxy;
n and m have independently a value selected from 0, 1 and 2; or a solvate or a salt thereof for use in the prevention or treatment of a condition associated with the alteration of the activity of GLB1.

In an embodiment (embodiment 2) the present invention relates to a compound for use as defined in embodiment 1 wherein each Ra or Rb independently represents, hydrogen, —C(═O)Rd, —SO₂Rd, —C₁₋₄ alkyl, —C₃₋₆ cycloalkyl, —C₅₋₁₀ aryl, —C₅₋₁₀ heteroaryl, or —C₃₋₇ heterocyclyl; said alkyl, cycloalkyl, aryl, heteroaryl or heterocyclyl groups optionally being substituted with 1, 2 or 3 groups independently selected from halogen, hydroxy, amine, —C₁₋₄ alkyl, —CN, methoxy, substituted aryl, substituted heteroaryl, -haloC₁₋₄alkyl, -dihaloC₁₋₄alkyl, -trihaloC₁₋₄alkyl, halomethoxy, dihalomethoxy and trihalomethoxy.

In an embodiment (embodiment 3) the present invention relates to a compound for use as defined in anyone of embodiments 1 and 2 of the present invention wherein G is a group —C(═O).

In another embodiment (embodiment 4) the present invention relates to a compound for use as defined in anyone of embodiments 1 to 3 of the present invention wherein R₄ is selected from chloro, bromo and —CF₃.

In another embodiment (embodiment 5) the present invention relates to a compound for use as defined in anyone of embodiments 1 to 4 of the present invention wherein R₁ is selected from hydrogen, halogen, —ORa and —C₁₋₄ alkyl optionally substituted with 1, 2 or 3 groups independently selected from halogen, hydroxy, —C₁₋₄ alkyl, —N(Rb)₂, methoxy, -haloC₁₋₄alkyl, -dihaloC₁₋₄alkyl, -trihaloC₁₋₄alkyl, halomethoxy, dihalomethoxy, and trihalomethoxy.

In another embodiment (embodiment 6) the present invention relates to a compound for use as defined in anyone of embodiments 1 to 5 of the present invention wherein Ra and Rb are independently selected from —C₁₋₄ alkyl, —C₃₋₆ cycloalkyl, —C₅₋₁₀ aryl, —C₅₋₁₀ heteroaryl, and —C₃₋₇ heterocyclyl; said alkyl, cycloalkyl, aryl, heteroaryl, or heterocyclyl groups optionally being substituted with 1, 2 or 3 groups independently selected from halogen, hydroxy, amine, —C₁₋₄ alkyl, —CN, methoxy, substituted aryl, substituted heteroaryl, -haloC₁₋₄alkyl, -dihaloC₁₋₄alkyl, -trihaloC₁₋₄alkyl, halomethoxy, dihalomethoxy and trihalomethoxy.

In another embodiment (embodiment 7) the present invention relates to a compound for use as defined in anyone of embodiments 1 to 6 of the present invention wherein Rc and Rd are independently selected from —C₁₋₄ alkyl, —C₅₋₁₀ aryl, and —C₃₋₇ heterocyclyl; said alkyl, cycloalkyl, aryl or heterocyclyl groups optionally being substituted with 1, 2 or 3 groups independently selected from halogen, hydroxy, methoxy, halomethoxy, dihalomethoxy, and trihalomethoxy.

In another embodiment (embodiment 8) the present invention relates to a compound for use as defined in anyone of embodiments 1 to 7 of the present invention wherein R₃ is selected from hydrogen, halogen, —ORa′ and —C₁₋₄ alkyl.

In another embodiment (embodiment 9) the present invention relates to a compound for use as defined in anyone of embodiments 1 to 8 of the present invention wherein Ra′ and Rb′ are independently selected from —C₁₋₄ alkyl, —C₃₋₆ cycloalkyl, —C₅₋₁₀ aryl, and —C₃₋₇ heterocyclyl; said alkyl, cycloalkyl, aryl or heterocyclyl groups optionally being substituted with 1, 2 or 3 groups independently selected from halogen, hydroxy, amine, methoxy, substituted aryl, substituted heteroaryl, halomethoxy, dihalomethoxy and trihalomethoxy.

In another embodiment (embodiment 10) the present invention relates to a compound for use as defined in anyone of embodiments 1 to 9 of the present invention wherein R₂ is independently selected from hydrogen, halogen and —C₁₋₄ alkyl.

In another embodiment (embodiment 11) the present invention relates to a compound for use as defined in anyone of embodiments 1 to 10 of the present invention wherein m represents 0 or 1.

In another embodiment (embodiment 12) the present invention relates to a compound for use as defined in anyone of embodiments 1 to 11 of the present invention wherein n represents 0 or 1.

Still in another embodiment (embodiment 13) the present invention relates to a compound for use as defined in anyone of embodiments 1 to 12 of the present invention wherein the condition associated with the alteration of the activity of GLB1 is selected from the group consisting of GM1 gangliosidoses and Morquio syndrome, type B.

Likewise, another aspect of the invention is the process for the preparation of a compound of general formula (I), or a solvate or a salt thereof.

In a second aspect the present invention relates to the use of a compound of general formula (I), or a salt or solvate thereof, in the preparation of a medicament for the prevention or treatment of a condition associated with the alteration of the activity of GLB1.

In a third aspect, the present invention is directed to a method for the prevention or treatment of a condition associated with the alteration of the activity of GLB1, which comprises the administration to a patient needing such prevention or treatment, of a therapeutically effective amount of at least one compound of general formula (I) or a salt or solvate thereof.

In a fourth aspect (also designated as embodiment 14) the present invention relates to a pharmaceutical composition comprising a compound of general formula (I), or a pharmaceutically acceptable salt or solvate thereof, and preferably at least one pharmaceutically acceptable excipient.

Finally, it is important to mention that a subgroup of compounds of formula (I), namely those of formula (Ia)

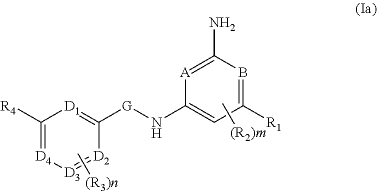

wherein:
A or B are independently selected from nitrogen and —CH═ with the proviso that at least one of A and B is a nitrogen atom;
D₁, D₂, D₃ and D₄ are nitrogen or ═CH— with the proviso that 0, 1 or 2 of D₁, D₂, D₃ and D₄ are nitrogen while the rest are —CH═;
G is selected from —C(═O)— and —SO₂—;
R₄ is an halogen atom;
R₁ is selected from hydrogen, halogen, hydroxy, —CN, —ORa, —SRa, —N(Rb)₂, —C₁₋₄ alkyl, —C(═O)ORc, —C(═O)N(Rb)₂, —SO₂N(Rb)₂, —C₃₋₆ cycloalkyl and —C₃₋₇ heterocyclyl; said alkyl, cycloalkyl and heterocyclyl groups are optionally substituted with 1, 2 or 3 groups independently selected from halogen, hydroxy, —C₁₋₄ alkyl, —N(Rb)₂, methoxy, -haloC₁₋₄alkyl, -dihaloC₁₋₄alkyl, -trihaloC₁₋₄alkyl, halomethoxy, dihalomethoxy, and trihalomethoxy;
each Ra or Rb independently represents, hydrogen, —C(═O)Rd, —SO₂Rd, —C₁₋₄ alkyl, —C₃₋₆ cycloalkyl, —C₅₋₁₀ aryl, —C₅₋₁₀ heteroaryl, or —C₃₋₇ heterocyclyl; said alkyl, cycloalkyl, aryl, heteroaryl or heterocyclyl groups optionally being substituted with 1, 2 or 3 groups independently selected from halogen, hydroxy, amine, —C₁₋₄ alkyl, —CN, methoxy, aryl, substituted heteroaryl, -haloC₁₋₄alkyl, -dihaloC₁₋₄alkyl, -trihaloC₁₋₄alkyl, halomethoxy, dihalomethoxy and trihalomethoxy;
each Rd or Rc independently represent hydrogen, —C₁₋₄ alkyl, —C₃₋₆ cycloalkyl, —C₅₋₁₀ aryl, —C₅₋₁₀ heteroaryl, or —C₃₋₇ heterocyclyl; said alkyl, cycloalkyl, aryl, heteroaryl or heterocyclyl groups optionally being substituted with 1, 2 or 3 groups independently selected from halogen, hydroxy, methoxy, halomethoxy, dihalomethoxy, and trihalomethoxy;
each R₃ is independently selected from hydrogen, halogen, hydroxy, —CN, —ORa′, —SRa′, —N(Rb′)₂ and —C₁₋₄ alkyl; said —C₁₋₄ alkyl group optionally being substituted with 1, 2 or 3 groups independently selected from halogen, hydroxy and —N(Rb)₂;
each Ra′ or Rb′ independently represent, on each occasion when used herein, hydrogen, —C(═O)Rd, —SO₂Rd, —C₁₋₄ alkyl, —C₃₋₆ cycloalkyl, —C₅₋₁₀ aryl, —C₅₋₁₀ heteroaryl, or —C₃₋₇ heterocyclyl; said alkyl, cycloalkyl, aryl, heteroaryl or heterocyclyl groups optionally being substituted with 1, 2 or 3 groups independently selected from halogen, hydroxy, amine, methoxy, substituted aryl, substituted heteroaryl, halomethoxy, dihalomethoxy and trihalomethoxy;
R₂ is selected from hydrogen and fluor;
n has a value selected from 0, 1 and 2;
m has a value selected from 0 and 1;
   are new products.

These new products of formula (Ia) as hereinabove described and the solvates and salts thereof constitute a fifth aspect of the invention (also designated as embodiment 15).

In an embodiment (embodiment 16 the present invention relates to a compound for use as defined in embodiment 15 wherein each Ra or Rb independently represents, hydrogen, —C(═O)Rd, —SO₂Rd, —C₁₋₄ alkyl, —C₃₋₆ cycloalkyl, —C₅₋₁₀ aryl, —C₅₋₁₀ heteroaryl, or —C₃₋₇ heterocyclyl; said alkyl, cycloalkyl, aryl, heteroaryl or heterocyclyl groups optionally being substituted with 1, 2 or 3 groups independently selected from halogen, hydroxy, amine, —C₁₋₄ alkyl, —CN, methoxy, substituted aryl, substituted heteroaryl, -haloC₁₋₄alkyl, -dihaloC₁₋₄alkyl, -trihaloC₁₋₄alkyl, halomethoxy, dihalomethoxy and trihalomethoxy.

In another embodiment (embodiment 17) the present invention relates to a compound as defined in anyone of embodiments 15 to 16 of the present invention wherein G is a group —C(═O).

In another embodiment (embodiment 18) the present invention relates to a compound as defined in anyone of embodiments 15 to 17 of the present invention wherein R₄ is selected from chloro and bromo.

In another embodiment (embodiment 19) the present invention relates to a compound as defined in anyone of embodiments 15 to 18 of the present invention wherein R₁ is selected from hydrogen, halogen, —ORa and —C₁₋₄ alkyl optionally substituted with 1, 2 or 3 groups independently selected from halogen, hydroxy, —C₁₋₄ alkyl, —N(Rb)₂, methoxy, -haloC₁₋₄alkyl, -dihaloC₁₋₄alkyl, -trihaloC₁₋₄alkyl, halomethoxy, dihalomethoxy, and trihalomethoxy.

In another embodiment (embodiment 20) the present invention relates to a compound as defined in anyone of embodiments 15 to 19 of the present invention wherein Ra and Rb are independently selected from —C₁₋₄ alkyl, —C₃₋₆ cycloalkyl, —C₅₋₁₀ aryl, —C₅₋₁₀ heteroaryl, and —C₃₋₇ heterocyclyl; said alkyl, cycloalkyl, aryl, heteroaryl, or heterocyclyl groups optionally being substituted with 1, 2 or 3 groups independently selected from halogen, hydroxy, amine, —C₁₋₄ alkyl, —CN, methoxy, substituted aryl, substituted heteroaryl, -haloC₁₋₄alkyl, -dihaloC₁₋₄alkyl, -trihaloC₁₋₄alkyl, halomethoxy, dihalomethoxy and trihalomethoxy.

In another embodiment (embodiment 21) the present invention relates to a compound as defined anyone of embodiments 15 to 20 of the present invention wherein Rc and Rd are independently selected from —C₁₋₄ alkyl, —C₅₋₁₀ aryl, and —C₃₋₇ heterocyclyl; said alkyl, cycloalkyl, aryl or heterocyclyl groups optionally being substituted with 1, 2 or 3 groups independently selected from halogen, hydroxy, methoxy, halomethoxy, dihalomethoxy, and trihalomethoxy.

In another embodiment (embodiment 22) the present invention relates to a compound as defined in anyone of embodiments 15 to 21 of the present invention wherein R₃ is selected from hydrogen, halogen, —ORa′ and —C₁₋₄ alkyl.

In another embodiment (embodiment 23) the present invention relates to a compound as defined in anyone of embodiments 15 to 22 of the present invention wherein Ra′ and Rb′ are independently selected from —C₁₋₄ alkyl, —C₃₋₆ cycloalkyl, —C₅₋₁₀ aryl, and —C₃₋₇ heterocyclyl; said alkyl, cycloalkyl, aryl or heterocyclyl groups optionally being substituted with 1, 2 or 3 groups independently selected from halogen, hydroxy, amine, methoxy, substituted aryl, substituted heteroaryl, halomethoxy, dihalomethoxy and trihalomethoxy.

In another embodiment (embodiment 24) the present invention relates to a compound as defined in anyone of embodiments 15 to 23 of the present invention wherein R₂ is independently selected from hydrogen, halogen and —C₁₋₄ alkyl.

In another embodiment (embodiment 25) the present invention relates to a compound as defined in anyone of embodiments 15 to 24 of the present invention wherein n represents 0 or 1.

Likewise, another aspect of the invention is the process for the preparation of a compound of general formula (I), or a solvate or a salt thereof.

### DETAILED DESCRIPTION OF THE INVENTION

According to an embodiment the invention relates to compounds of formula (Ib):

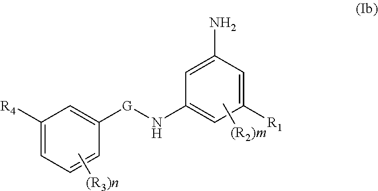

wherein n, m, G, R₁, R₂, R₃ and R₄ are as hereinabove defined or a solvate or a salt thereof for use in the prevention or treatment of a condition associated with the alteration of the activity of GLB1.

According to another embodiment the invention relates to compounds of formula (Ic):

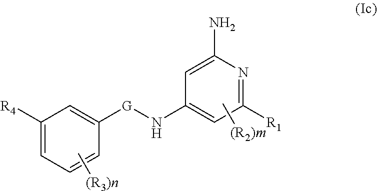

wherein n, m, G, R₁, R₂, R₃ and R₄ are as hereinabove defined or a solvate or a salt thereof for use in the prevention or treatment of a condition associated with the alteration of the activity of GLB1.

According to another embodiment the invention relates to compounds of formula (Id):

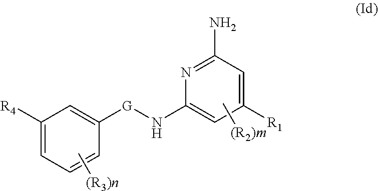

wherein n, m, G, R₁, R₂, R₃ and R₄ are as hereinabove defined or a solvate or a salt thereof for use in the prevention or treatment of a condition associated with the alteration of the activity of GLB1.

According to another embodiment the invention relates to compounds of formula (Ie):

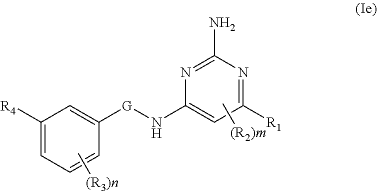

wherein n, m, G, R₁, R₂, R₃ and R₄ are as hereinabove defined or a solvate or a salt thereof for use in the prevention or treatment of a condition associated with the alteration of the activity of GLB1.

The compounds of formulae (I), (Ia), (Ib), (Ic), (Id) and (Ie) may contain chiral (asymmetric) centres or the molecule as a whole may be chiral. The individual stereoisomers (enantiomers and diastereoisomers) and mixtures of these are within the scope of the present invention.

According to one embodiment, in the compounds of formulae (I), (Ia), (Ib), (Ic), (Id) and (Ie) G is a group —C(═O)—.

According to another embodiment, in the compounds of formulae (I), (Ia), (Ib), (Ic), (Id) and (Ie), R₄ is selected from chloro, bromo and —CF₃. More preferably, R₄ is chloro.

According to another embodiment, in the compounds of formulae (I), (Ia), (Ib), (Ic), (Id) and (Ie), R₁ is selected from hydrogen, halogen, —ORa and —C₁₋₄ alkyl optionally substituted with 1, 2 or 3 groups independently selected from halogen, hydroxy, —C₁₋₄ alkyl, —N(Rb)₂, methoxy, -haloC₁₋₄alkyl, -dihaloC₁₋₄alkyl, -trihaloC₁₋₄alkyl, halomethoxy, dihalomethoxy, and trihalomethoxy. Preferred substituents for said alkyl groups are selected from halogen and —N(Rb)₂. More preferably, R₁ is selected from hydrogen and —ORa.

According to another embodiment, in the compounds of formulae (I), (Ia), (Ib), (Ic), (Id) and (Ie), Ra and Rb are independently selected from —C₁₋₄ alkyl, —C₃₋₆ cycloalkyl, —C₅₋₁₀ aryl, —C₅₋₁₀ heteroaryl, and —C₃₋₇ heterocyclyl; with optional substitution of those groups as described above. More preferably, Ra are Rb independently selected from —C₅₋₁₀ aryl, —C₅₋₁₀ heteroaryl and —C₃₋₇ heterocyclyl.

According to another embodiment, in the compounds of formulae (I), (Ia), (Ib), (Ic), (Id) and (Ie), Rc and Rd are independently selected from —C₁₋₄ alkyl, —C₅₋₁₀ aryl, and —C₃₋₇ heterocyclyl; with optional substitution of those groups as described above. More preferably, Rc are Rd independently selected from —C₁₋₄ alkyl, and —C₃₋₇ heterocyclyl.

According to another embodiment, in the compounds of formulae (I), (Ia), (Ib), (Ic), (Id) and (Ie), R₃ is selected from hydrogen, halogen, —ORa′ and —C₁₋₄ alkyl. More preferably, R₃ is selected from hydrogen, chloro, fluoro and —ORa′. Preferred substituents for said alkyl groups are selected from halogen and —N(Rb)₂.

According to another embodiment, in the compounds of formulae (I), (Ia), (Ib), (Ic), (Id) and (Ie), Ra′ and Rb′ are independently selected from —C₁₋₄ alkyl, —C₃₋₆ cycloalkyl, —C₅₋₁₀ aryl, and —C₃₋₇ heterocyclyl; with optional substitution of those groups as described above. More preferably, Ra′ are Rb′ independently selected from —C₁₋₄ alkyl and —C₃₋₆ cycloalkyl.

According to another embodiment, in the compounds of formulae (I), (Ia), (Ib), (Ic), (Id) and (Ie), R₂ is independently selected from hydrogen, halogen and —C₁₋₄ alkyl. More preferably, R₂ is selected from hydrogen and fluoro. Preferred substituents for said alkyl groups are selected from halogen and —N(Rb)₂.

According to another embodiment, in the compounds of formulae (I), (Ia), (Ib), (Ic), (Id) and (Ie), m represents 0 or 1.

According to another embodiment, in the compounds of formulae (I), (Ia), (Ib), (Ic), (Id) and (Ie), n represents 0 or 1.

In a further aspect the invention is directed to a compound selected from the group consisting of:
N-(2-aminopyrimidin-4-yl)-3-chloro-4-methoxybenzamide,
N-(2-aminopyrimidin-4-yl)-3-chloro-4-fluorobenzamide,
N-(6-aminopyridin-2-yl)-3-chlorobenzamide,
N-(2-aminopyrimidin-4-yl)-3-chlorobenzamide, and
N-(2-aminopyridin-4-yl)-3-chlorobenzamide,
   or a solvate or a salt thereof.

The compounds of formula (I) can be in the form of solvates or salts, preferably wherein the solvating agents and/or the salt's counter-ions are pharmaceutically acceptable species.

As used herein, the terms “Halogen” or “halo” refer to —F, —Cl, —Br or —I.

As used herein, the term “hydroxyl” refers to the group —OH,

As used herein, the term “alkyl” refers to a linear or branched hydrocarbon chain radical consisting of carbon and hydrogen atoms, containing no unsaturation, having the carbon atoms indicated in each case, which is attached to the rest of the molecule by a single bond. Exemplary alkyl groups can be methyl, ethyl, n-propyl, or i-propyl,

As used herein, the term “cycloalkyl” embraces saturated carbocyclic radicals and, unless otherwise specified, a cycloalkyl radical typically has from 3 to 6 carbon atoms. Examples include cyclopropyl, cyclobutyl, cyclopentyl, cyclohexyl and cycloheptyl. It is preferably cyclopropyl, cyclopentyl and cyclohexyl.

As used herein, the terms “heterocyclyl” or “heterocyclic group” embrace typically a non-aromatic, saturated or unsaturated C₃₋₇ carbocyclic ring, such as a 5, 6 or 7 membered radical, in which one or more, for example 1, 2, 3 or 4 of the carbon atoms preferably 1 or 2 of the carbon atoms are replaced by a heteroatom selected from N, O and S.

Saturated heterocyclyl radicals are preferred. A heterocyclic radical may be a single ring or two or more fused rings wherein at least one ring contains a heteroatom. When a heterocyclyl radical carries one or more substituents, the substituents may be the same or different.

A said optionally substituted heterocyclyl is typically unsubstituted or substituted with 1, 2 or 3 substituents which may be the same or different. Examples of heterocyclic radicals include piperidyl, pyrrolidyl, pyrrolinyl, piperazinyl, morpholinyl, thiomorpholinyl, pyrazolinyl, pyrazolidinyl, quinuclidinyl, tetrazolyl, cromanyl, isocromanyl, imidazolidinyl, oxiranyl, azaridinyl, 4,5-dihydro-oxazolyl and 3-aza-tetrahydrofuranyl.

As used herein, the term “haloC₁₋₄alkyl,” designates a C₁₋₄alkyl group wherein one of the hydrogen atoms has been replaced with a halogen atom.

As used herein, the term “dihaloC₁₋₄alkyl,” designates a C₁₋₄alkyl group wherein two of the hydrogen atoms have been replaced with a halogen atom. The hydrogen atoms replaced by halogens may be attached to the same carbon atom or to different carbon atoms.

As used herein, the term “trihaloC₁₋₄alkyl,” designates a C₁₋₄alkyl group wherein three of the hydrogen atoms have been replaced with a halogen atom. The hydrogen atoms replaced by halogens may be attached to the same carbon atom or to different carbon atoms.

As used herein, the term “aryl” designates typically a C₅₋₁₀ monocyclic or polycyclic aryl radical such as phenyl and naphthyl. Phenyl is preferred. A said optionally substituted aryl radical is typically unsubstituted or substituted with 1, 2 or 3 substituents which may be the same or different. The substituents are preferably selected from halogen atoms, preferably fluorine atoms, hydroxy groups, alkoxycarbonyl groups in which the alkyl moiety has from 1 to 4 carbon atoms, hydroxycarbonyl groups, carbamoyl groups, nitro groups, cyano groups, C₁₋₄ alkyl groups, C₁₋₄ alkoxy groups and C₁₋₄ hydroxyalkyl groups. When an aryl radical carries 2 or more substituents, the substituents may be the same or different. Unless otherwise specified, the substituents on an aryl group are typically themselves unsubstituted.

As used herein, the term “amine” refers to the group —NReRf, wherein Re and Rf are independently selected from H and C₁₋₄alkyl, wherein alkyl is as defined above. Preferably, amine refers to a NH₂ group.

As used herein, the term “heteroaryl” designates typically a 5- to 14-membered ring system, preferably a 5- to 10-membered ring system, comprising at least one heteroaromatic ring and containing at least one heteroatom selected from O, S and N.

A heteroaryl group may be a single ring or two or more fused rings wherein at least one ring contains a heteroatom. A said optionally substituted heteroaryl group is typically unsubstituted or substituted with 1, 2 or 3 substituents which may be the same or different. The substituents are preferably selected from halogen atoms, preferably fluorine, chlorine or bromine atoms, alkoxycarbonyl groups in which the alkyl moiety has from 1 to 4 carbon atoms, nitro groups, hydroxy groups, C₁₋₄ alkyl groups and C₁₋₄ alkoxy groups. When an heteroaryl radical carries 2 or more substituents, the substituents may be the same or different. Unless otherwise specified, the substituents on a heteroaryl radical are typically themselves unsubstituted.

Examples include pyridyl, pyrazinyl, pyrimidinyl, pyridazinyl, furyl, benzofuranyl, oxadiazolyl, oxazolyl, isoxazolyl, benzoxazolyl, imidazolyl, benzimidazolyl, thiazolyl, thiadiazolyl, thienyl, pyrrolyl, pyridinyl, benzothiazolyl, indolyl, indazolyl, purinyl, quinolyl, isoquinolyl, phthalazinyl, naphthyridinyl, quinoxalinyl, quinazolinyl, quinolizinyl, cinnolinyl, triazolyl, indolizinyl, indolinyl, isoindolinyl, isoindolyl, imidazolidinyl, pteridinyl, thianthrenyl, pyrazolyl, 2H-pyrazolo[3,4-d]pyrimidinyl, 1H-pyrazolo[3,4-d]pyrimidinyl, thieno[2,3-d]pyrimidinyl and the various pyrrolopyridyl radicals.

The mention of optionally substituted heteroaryl radicals or rests within the present invention is intended to cover the N-oxides obtainable from these radicals when they comprise N-atoms.

The term “pharmaceutically acceptable species” refers to compositions and molecular entities that are physiologically tolerable and do not typically produce an allergic reaction or a similar unfavorable reaction as gastric disorders, dizziness and suchlike, when administered to a human or animal. Preferably, the term “pharmaceutically acceptable” means it is approved by a regulatory agency of a state or federal government or is included in the U.S. Pharmacopoeia or other generally recognized pharmacopoeia for use in animals, and more particularly in humans.

The term “solvate” means any form of the active compound of the invention which has another molecule (for example a polar solvent such as water or ethanol, a cyclodextrin or a dendrimer) attached to it through noncovalent bonds. Methods of solvation are known within the art.

The invention also provides salts of the compounds of the invention. Non-limiting examples are sulphates; hydrohalide salts; phosphates; lower alkane sulphonates; arylsulphonates; salts of C₁₋₂₀ aliphatic mono-, di- or tribasic acids which may contain one or more double bonds, an aryl nucleus or other functional groups such as hydroxy, amino, or keto; salts of aromatic acids in which the aromatic nuclei may or may not be substituted with groups such as hydroxyl, lower alkoxyl, amino, mono- or di-lower alkylamino sulphonamido. Also included within the scope of the invention are quaternary salts of the tertiary nitrogen atom with lower alkyl halides or sulphates, and oxygenated derivatives of the tertiary nitrogen atom, such as the N-oxides. In preparing dosage formulations, those skilled in the art will select the pharmaceutically acceptable salts.

Solvates and salts can be prepared by methods known in the state of the art. Note that the non-pharmaceutically acceptable solvates also fall within the scope of the invention because they can be useful in preparing pharmaceutically acceptable salts and solvates.

The compounds of the invention also seek to include compounds that differ only in the presence of one or more isotopically enriched atoms. For example, compounds having the present structures except for the replacement of a hydrogen by a deuterium or tritium, or the replacement of a carbon by a carbon enriched in ¹¹C, ¹³C or ¹⁴C or the replacement of a nitrogen by a ¹⁵N enriched nitrogen are within the scope of this invention.

### Synthesis of Compounds of Formula (I)

Another aspect of the invention refers to procedures to obtain compounds of general formula (I). The following methods describe the procedures for obtaining compounds of general formula (I), or solvates or salts thereof.

Various synthetic routes for synthetizing compounds of formula (I) are summarized in the scheme below:

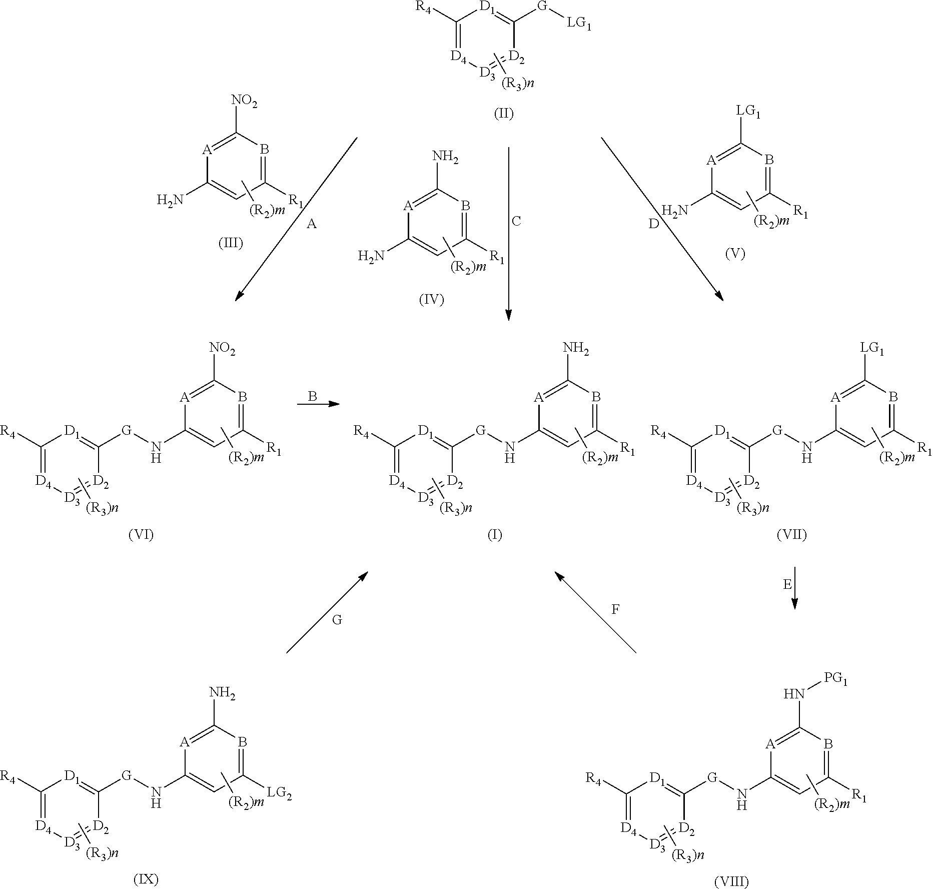

### Method 1

### Step 1

In a first method according to the invention a compound of formula (II), wherein D₁, D₂, D₃, D₄, G, R₃, R₄ and n are as defined in the first aspect of the invention, is reacted with a compound of formula (III), wherein A, B, R₁, R₂ and m are as defined in the first aspect of the invention, to yield a compound of formula (VI) as illustrated in reaction A of the scheme above.

Reaction A is carried out under standard amide coupling conditions, for example in the presence of a suitable coupling agent (e.g. 1,1′-carbonyldiimidazole, N,N′-cyclohexylcarbodiimide, 1-(3-dimethylaminopropyl)-3-ethylcarbodiimide (or hydrochloride thereof), N,N′-disuccinimidyl carbonate, benzotriazol-1-yloxytris(dimethylamino)phosphonium hexafluoro-phosphate, 2-(1H-benzotriazol-1-yl)-1,1,3,3-tetramethyluronium hexafluorophosphate (i.e. O-(1H-benzotriazol-1-yl)-N,N,N′,N′-tetramethyluronium hexafluorophosphate), benzotriazol-1-yloxytris-pyrrolidinophosphonium hexafluorophosphate, bromo-tris-pyrrolidinophosphonium hexafluorophosphate, 2-(1H-benzotriazol-1-yl)-1,1,3,3-tetramethyluronium tetra-fluorocarbonate, 1-cyclohexylcarbodiimide-3-propyloxymethyl polystyrene, O-(7-azabenzotriazol-1-yl)-N,N,N′,N′-tetramethyluronium hexafluorophosphate, O-benzotriazol-1-yl-N,N,N′,N′-tetramethyluronium hexfluoroborate), optionally in the presence of a suitable base (e.g. sodium hydride, sodium bicarbonate, potassium carbonate, pyridine, triethylamine, dimethylaminopyridine, diisopropylamine, sodium hydroxide, potassium tert-butoxide and/or lithium diisopropylamide (or variants thereof) and an appropriate solvent (e.g tetrahydrofurane, pyridine, toluene, dichloromethane, chloroform, acetonitrile, dimethylformamide, trifluoromethylbenzene, dioxane or triethylamine). Such reactions may be performed in the presence of a further additive such as 1-hydroxybenzotriazole hydrate.

Alternatively, the carboxylic acid group may be converted under standard conditions to the corresponding acyl chloride (e.g. in the presence of SOCl₂ or oxalyl chloride), which acyl chloride is then reacted with a compound of formula III, for example under similar conditions to those mentioned above. Alternatively still, when a carboxylic acid ester group is converted to a carboxylic acid amide, the reaction may be performed in the presence of a suitable reagent such as trimethylaluminium.

Alternatively a sulfonyl chloride of formula (II) wherein G is SO₂ and LG₁ is chloride may be reacted with the amines of formulae (III), (IV) or (V) under standard coupling conditions in a suitable solvent and in the presence of a suitable base. The aforementioned reaction may, in some case, yield products where two molecules of the sulfonyl chloride of formula (II) have reacted with the free amine group of the products of formulae (III), (IV) or (V) to yield disulfonated products. In such cases the compounds of interest of formulae (VI), (I) and (VII) are obtained by cleaving of the sulfonyl groups in a further reaction step.

The reaction mixture is stirred at low temperature or room temperature, or heated until the starting materials have been consumed. The reaction may be carried out with protecting groups present and those protecting groups may be removed after the reaction. Suitable protecting groups are known to the person skilled in the art (see T. W. Greene, “Protective Groups in Organic Synthesis”, 3rd Edition, New York, 1999)

### Step 2

The nitro group of the compound of formula (VI) is subsequently reduced to a primary amine group to yield the compound of formula (I) according to the invention as illustrated in reaction B of the scheme above. Reaction B is carried out with a suitable reducing agent such as Fe, SnCl₂, Raney Nickel and H₂/PtO₂. The reaction may be carried out in the presence of and acid such as acetic acid and in a suitable solvent such as ethyl acetate, water, methanol, ethanol and/or tetrahydrofurane. Other reducing agents or acids may be employed, as is known by the person skilled in the art. The reaction mixture is stirred at room temperature, or heated until the starting materials have been consumed. The reaction may be carried out with protecting groups present and those protecting groups may be removed after the reaction. Suitable protecting groups are known to the person skilled in the art (see T. W. Greene, “Protective Groups in Organic Synthesis”, 3rd Edition, New York, 1999).

### Method 2

In a second method according to the invention a compound of formula (II), wherein D₁, D₂, D₃, D₄, G, R₃, R₄ and n are as defined in the first aspect of the invention, is reacted with a compound of formula (IV), wherein A, B, R₁, R₂ and m are as defined in the first aspect of the invention, to yield a compound of formula (I) according to the invention as illustrated in reaction C of the scheme above.

Reaction C is carried out under standard amide coupling conditions such as those explained for step 1 of method 1 described above.

### Method 3

### Step 1

In a third method according to the invention a compound of formula (II), wherein D₁, D₂, D₃, D₄, G, R₃, R₄ and n are as defined in the first aspect of the invention, is reacted with a compound of formula (V), wherein A, B, R₁, R₂ and m are as defined in the first aspect of the invention and LG₁ represents a suitable leaving group such as such as iodo, bromo, chloro or a sulphonate group (e.g. —OS(O)₂CF₃, —OS(O)₂CH₃ or —OS(O)₂PhMe), to yield a compound of formula (VII) as illustrated in reaction D of the scheme above.

Reaction D is carried out under standard amide coupling conditions such as those explained for step 1 of method 1 described above.

### Step 2

The leaving group LG₁ of the compound of formula (VII) is subsequently replaced by a group —NH-PG₁ wherein PG₁ is an amino protecting group such as methyl carbamate, tert-butyl carbamate, 9-fluorenylmethyl carbamate, benzyl carbamate, 2-(trimethylsilyl)ethyl carbamate, trifluoroacetamide, benzylamine, allylamine, tritylamine, trichloroacetyl, trifluoroacetyl, p-toluenesulfonyl or allyl carbamate to yield the compound of formula (I) according to the invention as illustrated in reaction E of the scheme above.

The reaction is carried out by causing compound of formula (VII) to react with a compound of formula PG₁-NH₂.

The reaction may be carried out under standard conditions in the presence of a suitable base (e.g. pyridine, triethylamine, dimethylaminopyridine, diisopropylamine, sodium hydroxide, or mixtures thereof), and appropriate solvent (e.g. pyridine, dichloromethane, chloroform, tetrahydrofuran, dimethylformamide, triethylamine, dimethylsulphoxide, water or mixtures thereof) and for example at around room temperature or above, or under microwave irradiation reaction conditions.

Optionally it may also be carried out in the presence of an appropriate metal catalyst (or a salt or complex thereof) such as Cu, Cu(OAc)₂, CuI (or CuI/diamine complex) copper tris(triphenyl-phosphine)bromide, Pd(OAc)₂, tris(dibenzylideneacetone)dipalladium (0) (Pd₂(dba)₃) or NiCl₂ and of optional additive such as Ph₃P, 2,2′-bis(diphenylphosphino)-1,1′-binaphthyl, xantphos, NaI or and appropriate crown ether such as 18-crown-6-benzene, in the presence of an appropriate base such as sodium hydride, triethylamine, pyridine, N,N′-dimethylethylenediamine, sodium carbonate, potassium carbonate, potassium phosphate, cesium carbonate, sodium tert-butoxide or potassium tert-butoxide (or a mixture thereof, optionally in the presence of 4A molecular sieves), in a suitable solvent (e.g. dichloromethane, dioxane, toluene, ethanol, isopropanol, dimethylformamide, ethylene glycol, ethylene glycol dimethyl ether, water, dimethylsulfoxide, acetonitrile, dimethylacetamide, N-methylpyrrolidinone, tetrahydrofuran or a mixture thereof).

This reaction may be carried out under microwave irradiation reaction conditions

The reaction mixture may be stirred at room temperature or heated until the starting materials have been consumed. The reaction may be carried out with protecting groups present and those protecting groups may be removed after the reaction. Suitable protecting groups are known to the person skilled in the art (see T. W. Greene, “Protective Groups in Organic Synthesis”, 3rd Edition, New York, 1999).

### Step 3

In a final step, the amino protecting group (PG₁) of the compound of formula (VII) is cleaved to yield the compound of formula (I) as illustrated in reaction F of the scheme above.

Said reaction may be carried under standard conditions known to the person skilled in the art (see T. W. Greene, “Protective Groups in Organic Synthesis”, 3rd Edition, New York, 1999). For instance, the removal of a tert-butyl carbamate amine protecting group can be performed in the presence of a strong protic acid (e.g. 3M HCl or CF₃COOH) or TMS-I; a 2-(trimethylsilyl)ethyl carbamate amine protecting group can be removed in the presence of a fluoride ion (e.g. Bu₄NF); a 9-fluorenylmethyl carbamate amine protecting group by treatment with a mild base (e.g piperidine or morpholine); a benzyl carbamate amine protecting group by hydrogenolysis, treatment with BBr₃ or Na/NH₃, PdCl₂ and Et₃SiH; a trifluoroacetamide amine protecting group can be removed by treatment with a base (e.g. K₂CO₃) or NH₃; p-toluenesulfonyl protecting group can be cleaved with a strong acid or Na(Hg); allyl carbamate amine protecting group is cleaved with Pd(O) and a reducing agent (e.g. Bu₃SnH or Et₃SiH); benzylamine can be cleaved by hydrogenolysis (e.g. H₂, Pd/C and HCl); a tritylamine can be cleaved with HCl or H₂, Pd/C; an allylamine can be cleaved by treatment with polymethylhydrosiloxane (PMHS), ZnCl₂ and Pd(PPh₃)₄ or in oxydative conditions (e. g. NMO, OsO₄ and NaIO₄); a trichloroacetyl amine protecting group can be removed with NaBH₄; a trifluoroacetyl amine protecting group can be cleaved with a base (e.g. K₂CO₃ Na₂CO₃)

Other deprotection conditions may be employed, as is known by the person skilled in the art. An appropriate solvent may be used. The reaction mixture is stirred at room temperature, or heated until the starting materials have been consumed.

### Method 4

In a fourth method according to the invention a compound of formula (IX), wherein A, B, D₁, D₂, D₃, D₄, G, R₂, R₃, R₄, m and n are as defined in the first aspect of the invention is caused to reacted to yield a compound of formula (I) according to the invention as illustrated in reaction G of the scheme above.

Compounds wherein R₁ represents a group selected from —ORa, SRa and —N(Rb)₂

To prepare compounds of formula (I) wherein R₁ represents a group selected from —ORa, SRa and —N(Rb)₂ a compound of formula (IX) wherein LG₂ represents a leaving group such as iodo, bromo, chloro or a sulphonate group (e.g. —OS(O)₂CF₃, —OS(O)₂CH₃ or —OS(O)₂PhMe) is caused to react with a compound of formula H—R₁ wherein R₁ represents —ORa, —SRa or —N(Rb)₂ and Ra and Rb are as defined above. Said reaction may be performed under standard conditions in the presence of a suitable base such as pyridine, triethylamine, dimethylaminopyridine, diisopropylamine, sodium hydroxide, or mixtures thereof), and an appropriate solvent such as pyridine, dichloromethane, chloroform, tetrahydrofuran, dimethylformamide, triethylamine, dimethylsulphoxide, water or mixtures thereof and, for example, at around room temperature or above, or under microwave irradiation reaction conditions.

The reaction may also be carried out in the presence of an appropriate metal catalyst (or a salt or complex thereof) such as Cu, Cu(OAc)₂, CuI (or CuI/diamine complex) copper tris(triphenyl-phosphine)bromide, Pd(OAc)₂, tris(dibenzylideneacetone) dipalladium (0) (Pd₂(dba)₃) or NiCl₂ and also optionally in the presence of an additive such as Ph₃P, 2,2′-bis(diphenylphosphino)-1,1′-binaphthyl, xantphos, NaI or and appropriate crown ether such as 18-crown-6-benzene, in the presence of an appropriate base such as sodium hydride, triethylamine, pyridine, N,N′-dimethylethylenediamine, sodium carbonate, potassium carbonate, potassium phosphate, cesium carbonate, sodium tert-butoxide or potassium tert-butoxide (or a mixture thereof, optionally in the presence of 4A molecular sieves), in a suitable solvent (e.g. dichloromethane, dioxane, toluene, ethanol, isopropanol, dimethylformamide, ethylene glycol, ethylene glycol dimethyl ether, water, dimethylsulfoxide, acetonitrile, dimethylacetamide, N-methylpyrrolidinone, tetrahydrofuran or a mixture thereof.

This reaction may be carried out under microwave irradiation reaction conditions

The reaction mixture may be stirred at room temperature or heated until the starting materials have been consumed. The reaction may be carried out with protecting groups present and those protecting groups may be removed after the reaction. Suitable protecting groups are known to the person skilled in the art (see T. W. Greene, “Protective Groups in Organic Synthesis”, 3rd Edition, New York, 1999).

Compounds wherein R₁ represents a group selected from represents —C₁₋₄ alkyl, —C₃₋₆ cycloalkyl and —C₃₋₇ heterocyclyl.

To prepare compounds of formula (I) wherein R₁ represents a group selected from represents —C₁₋₄ alkyl, —C₃₋₆ cycloalkyl and —C₃₋₇ heterocyclyl, a compound of formula (IX) wherein LG₂ represents a suitable leaving group such as iodo, bromo, chloro or a sulphonate group (e.g. —OS(O)₂CF₃, —OS(O)₂CH₃ or —OS(O)₂PhMe) is caused to react with a compound of formula Q-R₁ wherein Q represents a suitable group such as alkali metal group (e. g. lithium), a Grignard reagent (e.g. MgX), —B(OH)₂, B(OR)₂ or —Sn(R)₃, wherein each R independently represents an alkyl group, or, in the case of —B(OR)₂, the respective R groups may be linked together to form a 4- to 6-membered cyclic group. The reaction may be performed, for example in the presence of a suitable catalyst system, e.g. a metal (or a salt or complex thereof) such as Pd, Cu, Pd/C, PdCl₂, Pd(OAc)₂, Pd(Ph₃P)₄, Pd(Ph₃P)₂Cl₂ (i.e. palladium tetrakistriphenylphosphine), Pd₂(dba)₃ or NiCl₂ and a ligand such as t-Bu₃P, (C₆H₁₁)₃P, Ph₃P, AsPh₃, P(o-Tol)₃, 1,2-bis(diphenylphosphino)ethane, 2,2′-bis(di-tert-butylphosphino)-1,1′-biphenyl, xantphos or a mixture thereof, together with a suitable base such as, sodium carbonate, potassium phosphate, cesium carbonate, sodium hydroxide, potassium hydroxide, potassium carbonate, cesium fluoride, triethylamine, diisopropylethylamine, sodium tert-butoxide, or potassium tert-butoxide (or mixtures thereof) in a suitable solvent such as dioxane, toluene, ethanol, dimethylformamide, ethylene glycol dimethyl ether, water, dimethylsulfoxide, acetonitrile, dimethylacetamide, N-methylpyrrolidinone, tetrahydrofuran or mixtures thereof. The reaction may also be carried out for example at room temperature or above. Alternative reactions conditions include microwave irradiation conditions.

The initial compounds and starting materials, e.g. the compounds of formula (II), (III), (IV) are either commercially available or can be obtained following procedures described in the literature. Compounds of formula (IX) can also be obtained following anyone of methods 1, 2 or 3 described above.

### Method 5

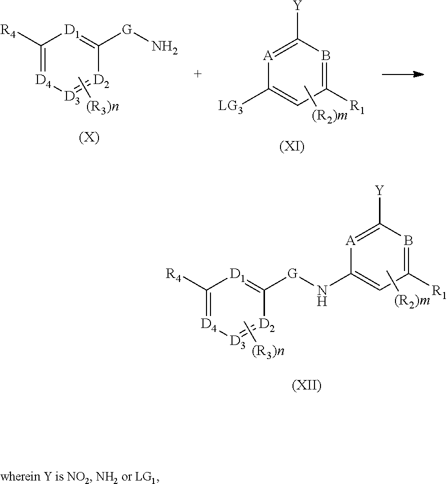

In a fifth method according to the invention a compound of formula (X), wherein D₁, D₂, D₃, D₄, G, R₃, R₄, and n are as defined in the first aspect of the invention is caused to react with a compound of formula (XI) wherein A, B, R₁, R₂ and m are as defined in the first aspect of the invention, Y is selected from the group consisting of NH₂, NO₂ and LG₁, and LG₃ is a suitable leaving group such as such as iodo, bromo, chloro or a sulphonate group (e.g. —OS(O)₂CF₃, —OS(O)₂CH₃ or —OS(O)₂PhMe) to yield a compound of formula (XII). When Y is NH₂ the compound of formula (XII) corresponds to a compound of formula (I) according to the invention. When Y is NO₂ the compound of formula (XII) corresponds to a compound of formula (VI) which can further be converted into a compound according to the invention through the reaction B explained above. When Y is a group LG₁ such as iodo, bromo, chloro or a sulphonate group (e.g. —OS(O)₂CF₃, —OS(O)₂CH₃ or —OS(O)₂PhMe), the compound of formula (XII) corresponds to a compound of formula (VII) which can further be converted into a compound according to the invention through the reactions E and F as explained above.

The above mentioned reaction may be performed under standard metal-catalysed cross coupling conditions in the presence of an appropriate metal catalyst (or a salt or complex thereof) such as Cu, Cu(OAc)₂, CuI (or CuI/diamine complex) copper tris(triphenyl-phosphine)bromide, Pd(OAc)₂, tris(dibenzylideneacetone)dipalladium (0) (Pd₂(dba)₃) or NiCl₂ and of optional additive such as Ph₃P, 2,2′-bis(diphenylphosphino)-1,1′-binaphthyl, Xantphos, NaI or and appropriate crown ether such as 18-crown-6-benzene, in the presence of an appropriate base such as sodium hydride, triethylamine, pyridine, N,N′-dimethylethylenediamine, sodium carbonate, potassium carbonate, potassium phosphate, cesium carbonate, sodium tert-butoxide or potassium tert-butoxide or a mixture thereof, optionally in the presence of 4A molecular sieves, in a suitable solvent (e.g. dichloromethane, dioxane, toluene, ethanol, isopropanol, dimethylformamide, ethylene glycol, ethylene glycol dimethyl ether, water, dimethylsulfoxide, acetonitrile, dimethylacetamide, N-methylpyrrolidinone, tetrahydrofuran or a mixture thereof). The reaction may be carried out under microwave irradiation reaction conditions

The reaction mixture may be stirred at room temperature or heated until the starting materials have been consumed. The reaction may be carried out with protecting groups present and those protecting groups may be removed after the reaction. Suitable protecting groups are known to the person skilled in the art (see T. W. Greene, “Protective Groups in Organic Synthesis”, 3rd Edition, New York, 1999).

### Use of the Compounds of the Invention

The compounds of general formula (I) are useful for the treatment of and/or prevention of conditions associated with the alteration of the activity of beta galactosidase, specially galactosidase beta-1 or GLB1, including GM1 gangliosidoses and Morquio syndrome, type B.

Therefore, in another aspect the invention is directed to a compound of formula (I)

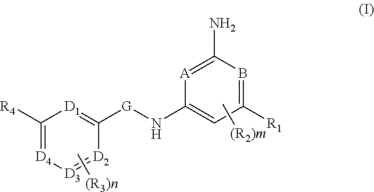

wherein:
A or B are independently selected from nitrogen and —CH═;
D₁, D₂, D₃ and D₄ are nitrogen or ═CH— with the proviso that 0, 1 or 2 of D₁, D₂, D₃ and D₄ are nitrogen while the rest are —CH═;
G is selected from —C(═O)— and —SO₂—;
R₄ is selected from halogen, methyl, —CF₃ and —OCF₃;
R₁ is selected from hydrogen, halogen, hydroxy, —CN, —ORa, —SRa, —N(Rb)₂, —C₁₋₄ alkyl, —C(═O)ORc, —C(═O)N(Rb)₂, —SO₂N(Rb)₂, —C₃₋₆ cycloalkyl and —C₃₋₇ heterocyclyl; said alkyl, cycloalkyl and heterocyclyl groups are optionally substituted with 1, 2 or 3 groups independently selected from halogen, hydroxy, —C₁₋₄ alkyl, —N(Rb)₂, methoxy, -haloC₁₋₄alkyl, -dihaloC₁₋₄alkyl, -trihaloC₁₋₄alkyl, halomethoxy, dihalomethoxy, and trihalomethoxy;
each Ra or Rb independently represents, hydrogen, —C(═O)Rd, —SO₂Rd, —C₁₋₄ alkyl, —C₃₋₆ cycloalkyl, —C₅₋₁₀ aryl, —C₅₋₁₀ heteroaryl, or —C₃₋₇ heterocyclyl; said alkyl, heteroaryl, cycloalkyl, aryl or heterocyclyl groups optionally being substituted with 1, 2 or 3 groups independently selected from halogen, hydroxy, amine, —C₁₋₄ alkyl, —CN, methoxy, aryl, substituted heteroaryl, -haloC₁₋₄alkyl, -dihaloC₁₋₄alkyl, -trihaloC₁₋₄alkyl, halomethoxy, dihalomethoxy and trihalomethoxy;
each Rd or Rc independently represent hydrogen, —C₁₋₄ alkyl, —C₃₋₆ cycloalkyl, —C₅₋₁₀ aryl, —C₅₋₁₀ heteroaryl, or —C₃₋₇ heterocyclyl; said alkyl, cycloalkyl, aryl, heteroaryl or heterocyclyl groups optionally being substituted with 1, 2 or 3 groups independently selected from halogen, hydroxy, methoxy, halomethoxy, dihalomethoxy, and trihalomethoxy;
each R₃ is independently selected from hydrogen, halogen, hydroxy, —CN, —ORa′, —SRa′, —N(Rb′)₂ and —C₁₋₄ alkyl; said —C₁₋₄ alkyl group optionally being substituted with 1, 2 or 3 groups independently selected from halogen, hydroxy and —N(Rb)₂;
each Ra′ or Rb′ independently represent, on each occasion when used herein, hydrogen, —C(═O)Rd, —SO₂Rd, —C₁₋₄ alkyl, —C₃₋₆ cycloalkyl, —C₅₋₁₀ aryl, —C₅₋₁₀ heteroaryl, or —C₃₋₇ heterocyclyl; said alkyl, cycloalkyl, aryl, heteroaryl or heterocyclyl groups optionally being substituted with 1, 2 or 3 groups independently selected from halogen, hydroxy, amine, methoxy, substituted aryl, substituted heteroaryl, halomethoxy, dihalomethoxy and trihalomethoxy;
each R₂ is independently selected from hydrogen, halogen, hydroxy, —CN, —ORa, —SRa, —N(Rb)₂, —C₁₋₄ alkyl, —C₃₋₆ cycloalkyl, —C₅₋₁₀ aryl and —C₃₋₇ heterocyclyl; said alkyl, cycloalkyl, aryl or heterocyclyl groups optionally being substituted with 1, 2 or 3 groups independently selected from halogen, hydroxy, —C₁₋₄ alkyl, —N(Rb)₂, methoxy, -haloC₁₋₄alkyl, -dihaloC₁₋₄alkyl, -trihaloC₁₋₄alkyl, halomethoxy, dihalomethoxy, and trihalomethoxy;
n and m have independently a value selected from 0, 1 or 2;

or a solvate or a salt thereof for use in the prevention or treatment of a condition associated with the alteration of the activity of GLB1.

In a particular embodiment, the invention is directed to a compound of formula (I) as defined in above, or a pharmaceutically acceptable solvate or a salt, for the use in the treatment of a disease or condition selected from the group consisting of GM1 gangliosidoses and Morquio syndrome, type B.

Further preferred embodiments of compounds of formula (I) for use as a medicament are as defined previously herein.

### Pharmaceutical Compositions

The compounds of the present invention can be used with at least another drug to provide a combination therapy. This other drug or drugs may be part of the same composition, or may be provided as a separate composition and can be administered at the same time or at different times.

The term “treatment” or “treating” in the context of this document means administration of a compound or a formulation according to this invention to prevent, improve or eliminate the disease or one or more symptoms associated with the disease. “Treatment” also encompasses preventing, improving or eliminating the physiological sequelae of the disease.

The term “excipient” refers to a vehicle, diluent or adjuvant that is administered with the active ingredient. Such pharmaceutical excipients can be sterile liquids, such as water and oils, including those of petroleum, animal, vegetable or synthetic origin, such as peanut oil, soybean oil, mineral oil, sesame oil and similars. Water or saline aqueous solutions and aqueous dextrose and glycerol solutions, particularly for injectable solutions, are preferably used as vehicles. Suitable pharmaceutical vehicles are described in “Remington's Pharmaceutical Sciences” by E. W. Martin, 21^{st} Edition, 2005; or “Handbook of Pharmaceutical Excipients”, Rowe C. R.; Paul J. S.; Marian E. Q., sixth Edition.

Examples of pharmaceutical compositions include any solid composition (tablets, pills, capsules, granules, etc.) or liquid composition (solutions, suspensions or emulsions) for oral, topical or parenteral administration.

In a preferred embodiment the pharmaceutical compositions are in oral delivery form. Pharmaceutical forms suitable for oral administration may be tablets and capsules and may contain conventional excipients known in the art such as binders, for example syrup, gum arabic, gelatin, sorbitol, tragacanth or polyvinylpyrrolidone; fillers, for example lactose, sugar, cornstarch, calcium phosphate, sorbitol or glycine; lubricants for the preparation of tablets, for example magnesium stearate; disintegrants, for example starch, polyvinylpyrrolidone, sodium starch glycolate or microcrystalline cellulose; or pharmaceutically acceptable wetting agents such as sodium lauryl sulphate.

Solid oral compositions can be prepared by conventional methods of blending, filling or preparation of tablets. Repeated blending operations can be used to distribute the active ingredient in all the compositions that use large amounts of fillers. Such operations are conventional in the art. The tablets can be prepared, for example, by dry or wet granulation and optionally can be coated by well known methods in normal pharmaceutical practice, in particular using an enteric coating.

Pharmaceutical compositions can also be adapted for parenteral administration, such as sterile solutions, suspensions or lyophilized products in the appropriate unit dosage form. Suitable excipients such as fillers, buffering agents or surfactants can be used.

The mentioned formulations will be prepared using standard methods such as those described or referred to in the Spanish and U.S. Pharmacopoeias and similar reference texts.

In general, the effective amount of a compound of the invention to be administered will depend on the relative efficacy of the compound chosen, the severity of the disorder being treated and the patient's weight. However, the active compounds will normally be administered one or more times a day, for example 1, 2, 3 or 4 times daily, with typical total daily doses in the range from 0.01 up to 1000 mg/kg/day.

In order to facilitate the understanding of the preceding ideas, some examples of experimental procedures and embodiments of the present invention are described below. These examples are merely illustrative.

### EXAMPLES

### General Experimental Conditions

Hereinafter, the term “h” means hours, “eq” means equivalents, “min” means minutes, “Pd₂(dba)₃” means tris(dibenzylideneacetone)-dipalladium(0), “XantPhos” means 4,5-bis(diphenylphosphino)-9,9-dimethylxanthene; “EDCI.HCl” means 1-ethyl-3-(3-dimethylaminopropyl)carbodiimide hydrochloride; “HOBt” means hydroxybenzotriazole; “SnCl₂” means tin(II) chloride; “DPPA” means diphenylphosphoryl azide.

NMR spectra were recorded in a Varian Mercury 400 MHz spectrometer (at room temperature).

The HPLC measurements were performed using a HPLC Waters Alliance HT comprising a pump (Edwards RV12) with degasser, an autosampler, a diode array detector and a column as specified in the respective methods below. Flow from the column was split to a MS spectrometer. The MS detector was configured with an eletrospray ionization source (micromass ZQ4000), Nitrogen was used as the nebulizer gas. Data acquisition was performed with MassLynx software.

MW calculated is an isotopic average and the “found mass” is referring to the most abundant isotope detected in the LC-MS.

The reversed phase HPLC purifications were carried out on a YMC-Pack ODS-AQ (50×4.6 mm. D S. 3 μm, 12 nm). Solvent A: water 0.1% formic acid; Solvent B: acetonitrile with 0.1% formic acid. Gradient: 5% of B to 100% of B within 3.5 min. Flux: 1.6 mL/min at 50° C.

### Synthesis of Compounds of the Invention

### General Procedure I:

In a particular embodiment according to synthetic route A explained under the section “synthesis of compounds of formula (I) an aniline of formula (IIIa) and an acid halide of formula (IIa) are reacted to prepared intermediates of formula (VIa):

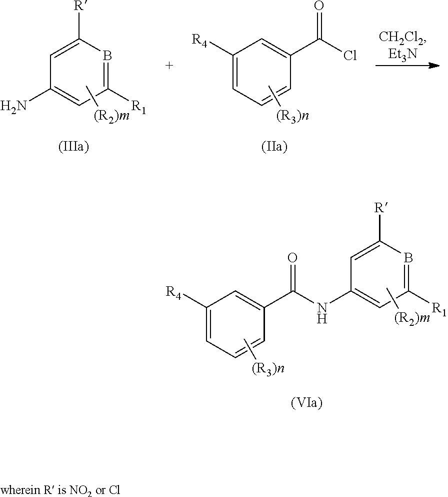

A solution of the appropriate aniline of formula (IIIa) (1eq.) (ex: 4-methyl-3-nitroaniline) and triethylamine (1 eq) in dry dichloromethane (4.5 mL/mmol) was cooled down to 0° C. under inert atmosphere. Then, the appropriate acid chloride of formula (IIa) (1eq) (ex: 3-chlorobenzoyl chloride) was added. After a period of 2 to 5 h, the solvent was removed under vacuum. The dry residue was taken up into ethyl acetate and washed with water. After extraction with ethyl acetate (3×), the combined organic layers were dried (magnesium sulphate), filtered and concentrated to yield the desired nitro intermediate (ex: 3-chloro-N-(4-methyl-3-nitrophenyl)benzamide). The crude was used as such for next step.

### Intermediate 1

### 3-Chloro-N-(2-fluoro-5-nitrophenyl)benzamide

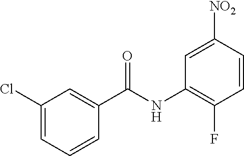 [SMILES: O=C(Nc1cc([N+](=O)[O-])ccc1F)c1cccc(Cl)c1]

HPLC-MS: Rt=3.10 min, (M+H)⁺ m/z 295.

Yield: 69%

### Intermediate 2

### 3-Chloro-N-(4-methyl-3-nitrophenyl)benzamide

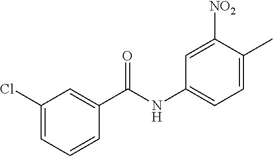 [SMILES: Cc1ccc(NC(=O)c2cccc(Cl)c2)cc1[N+](=O)[O-]]

HPLC-MS: Rt=3.12 min, (M+H)⁺ m/z 291.

Yield: 70%

### Intermediate 3

### 3-Chloro-N-(2-fluoro-4-methyl-5-nitrophenyl)benzamide

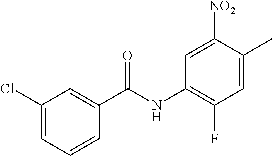 [SMILES: Cc1cc(F)c(NC(=O)c2cccc(Cl)c2)cc1[N+](=O)[O-]]

HPLC-MS: Rt=3.08 min, (M+H)⁺ m/z 309.

Yield: 94%

### Intermediate 4

### 3-Bromo-N-(3-nitrophenyl)benzamide

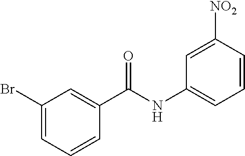 [SMILES: O=C(Nc1cccc([N+](=O)[O-])c1)c1cccc(Br)c1]

HPLC-MS: Rt=3.03 min, (M+H)⁺ m/z 321/323.

Yield: 89%

### Intermediate 5

### 3,5-Dichloro-N-(3-nitrophenyl)benzamide

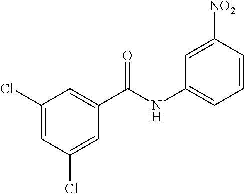 [SMILES: O=C(Nc1cccc([N+](=O)[O-])c1)c1cc(Cl)cc(Cl)c1]

HPLC-MS: Rt=2.47 min, (M+H)⁺ m/z 281,283.

Yield: 78%

### Intermediate 6

### 5-Chloro-N-(3-nitrophenyl)-2-(trifluoromethyl)benzamide

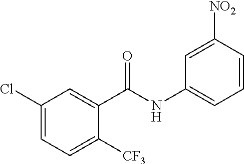 [SMILES: O=C(Nc1cccc([N+](=O)[O-])c1)c1cc(Cl)ccc1C(F)(F)F]

HPLC-MS: Rt=3.00 min, (M+H)⁺ m/z 345.

Yield: 61%

### Intermediate 7

### 2,5-Dichloro-N-(3-nitrophenyl)benzamide

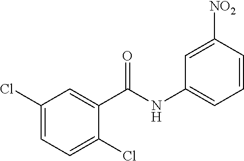 [SMILES: O=C(Nc1cccc([N+](=O)[O-])c1)c1cc(Cl)ccc1Cl]

HPLC-MS: Rt=2.98 min, (M+H)⁺ m/z 311,313.

Yield: 99%

### Intermediate 8

### 3-Chloro-N-(2-chloropyridin-4-yl)benzamide

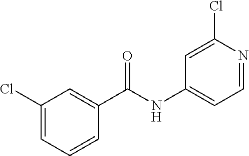 [SMILES: O=C(Nc1ccnc(Cl)c1)c1cccc(Cl)c1]

HPLC-MS: Rt=2.80 min, (M+H)⁺ m/z 267, 269.

¹H NMR (400 MHz, DMSO-d₆) δ 10.85 (s, 1H), 8.33 (d, J=5.6 Hz, 2H), 8.01 (t, J=1.8 Hz, 2H), 7.93 (d, J=1.9 Hz, 3H), 7.90 (m, 1H), 7.72 (m, 3H), 7.60 (t, J=7.9 Hz, 2H).

Yield: 91%

### Intermediate 9

### N-(2-Chloropyridin-4-yl)-3-(trifluoromethoxy)benzamide

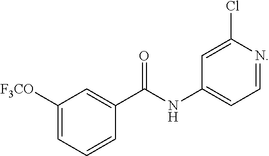 [SMILES: O=C(Nc1ccnc(Cl)c1)c1cccc(OC(F)(F)F)c1]

HPLC-MS: Rt=3.08 min, [M+H]⁺ m/z 317, 319.

Yield: 70%

### Intermediate 10

### 3-Chloro-N-(3-methyl-5-nitrophenyl)benzamide

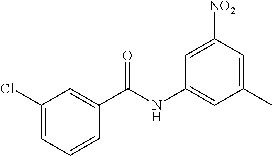 [SMILES: Cc1cc(NC(=O)c2cccc(Cl)c2)cc([N+](=O)[O-])c1]

HPLC-MS: Rt=3.38 min, [M+H]⁺ m/z 332, 334.

Yield: 99%.

### Intermediate 11

### 3-Chloro-N-(2-chloro-6-(pyridin-2-yloxy)pyridin-4-yl)benzamide

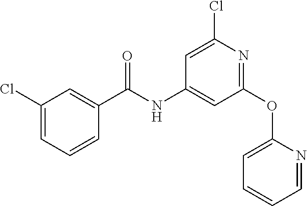 [SMILES: O=C(Nc1cc(Cl)nc(Oc2ccccn2)c1)c1cccc(Cl)c1]

HPLC-MS: Rt=3.18 min, (M+H)⁺ m/z 360, 362.

### Intermediate 12

### 3-Chloro-N-(2,6-dichloropyridin-4-yl)benzamide

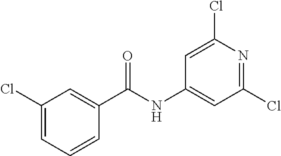 [SMILES: O=C(Nc1cc(Cl)nc(Cl)c1)c1cccc(Cl)c1]

HPLC-MS: Rt=3.43 min, (M+H)⁺ m/z 300, 302.

Yield: 24%.

### Example 1

### N-(3-amino-5-chlorophenyl)-3-chlorobenzamide

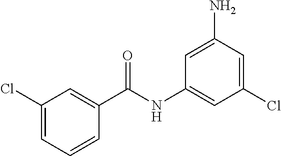 [SMILES: Nc1cc(Cl)cc(NC(=O)c2cccc(Cl)c2)c1]

HPLC-MS: Rt=2.92 min, (M+H)⁺ m/z 281, 283.

¹H NMR (400 MHz, CD₃OD) δ 7.91 (dd, J=2.8, 1.0 Hz, 1H), 7.82 (ddd, J=7.7, 1.7, 1.1 Hz, 1H), 7.58 (ddd, J=8.0, 2.1, 1.1 Hz, 1H), 7.48 (dd, J=11.8, 4.0 Hz, 1H), 7.04 (t, J=1.9 Hz, 1H), 7.01 (t, J=1.9 Hz, 1H), 6.50 (t, J=1.9 Hz, 1H).

Yield: 18%.

### General Procedure II:

In a particular embodiment according to synthetic routes A or C explained under the section “synthesis of compounds of formula (I) a compound of formula (IIIb) and an acid halide of formula (IIb) are reacted to prepared compounds of formula (VIb):

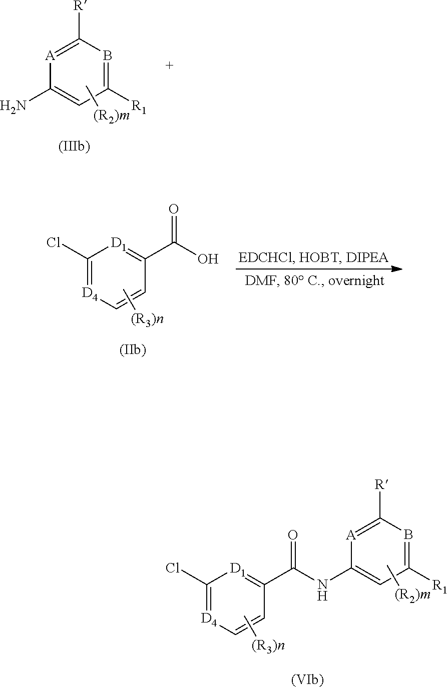

It must be noted that when R′ is an amino group the compounds of formula (VIb) are compounds of formula (I) according to the invention.

To a stirred solution of the appropriate acid (ex: 5-chloro-2-fluorobenzoic acid) (1 eq) in dimethylformamide (1.75 mL/mmol), EDCI.HCl (1.2 eq) and HOBt (1.2 eq) were added. After 20 min, the appropriate amine (ex: 3-nitroaniline) (1 eq) and diisopropylethylamine (2.5 eq) were added. The reaction mixture was stirred at room temperature (in some cases at 80° C.) overnight (in some cases up to three days). Volatiles were removed under vacuum and the crude mixture extracted with ethyl acetate (3×) washed with HCl (1M) (3×) and sodium bicarbonate (sat. sol.) (3×). The combined organic layers were dried (magnesium sulphate), filtered and concentrated in vacuum to obtain the desired amide intermediate which was used as such for the next step (ex: 5-chloro-2-fluoro-N-(3-nitrophenyl)benzamide).

In some cases the residue was purified by flash column chromatography (dichloromethane/methanol) to yield the desired amide product.

### Intermediate 13

### 5-Chloro-2-fluoro-N-(3-nitrophenyl)benzamide

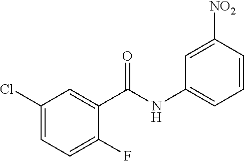 [SMILES: O=C(Nc1cccc([N+](=O)[O-])c1)c1cc(Cl)ccc1F]

HPLC-MS: Rt=2.95 min, (M+H)⁺ m/z 295, 297.

Yield: 44%

### Intermediate 14

### 5-Chloro-2-methoxy-N-(3-nitrophenyl)benzamide

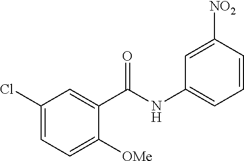 [SMILES: COc1ccc(Cl)cc1C(=O)Nc1cccc([N+](=O)[O-])c1]

HPLC-MS: Rt=3.13 min, [M+H]+m/z 307,309.

Yield: 46%

### Intermediate 15

### 3-Chloro-4-fluoro-N-(3-nitrophenyl)benzamide

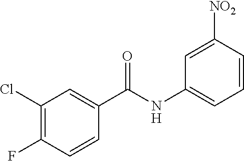 [SMILES: O=C(Nc1cccc([N+](=O)[O-])c1)c1ccc(F)c(Cl)c1]

HPLC-MS: Rt=2.87 min, [M+H]⁺ m/z 295,297.

Yield: 59%

### Intermediate 16

### 3-Chloro-2-methyl-N-(3-nitrophenyl)benzamide

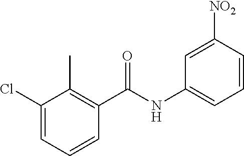 [SMILES: Cc1c(Cl)cccc1C(=O)Nc1cccc([N+](=O)[O-])c1]

HPLC-MS: Rt=2.95 min, [M+H]⁺ m/z 291, 293.

Yield: 25%

### Intermediate 17

### 3-Chloro-4-methoxy-N-(3-nitrophenyl)benzamide

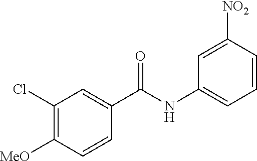 [SMILES: COc1ccc(C(=O)Nc2cccc([N+](=O)[O-])c2)cc1Cl]

HPLC-MS: Rt=2.93 min, [M+H]⁺ m/z 307, 309.

Yield: 53%

### Intermediate 18

### 3-Chloro-5-methoxy-N-(3-nitrophenyl)benzamide

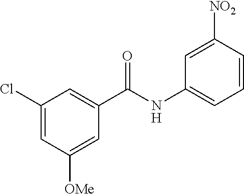 [SMILES: COc1cc(Cl)cc(C(=O)Nc2cccc([N+](=O)[O-])c2)c1]

HPLC-MS: Rt=3.07 min, [M+H]⁺ m/z 307, 309.

Yield: 57%

### Intermediate 19

### 2-Chloro-N-(3-nitrophenyl)isonicotinamide

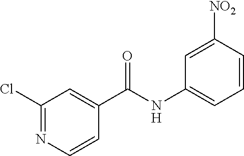 [SMILES: O=C(Nc1cccc([N+](=O)[O-])c1)c1ccnc(Cl)c1]

HPLC-MS: Rt=2.65 min, (M+H)⁺ m/z 278, 279.

Yield: 58%

### Intermediate 20

### 6-Chloro-N-(3-nitrophenyl)picolinamide

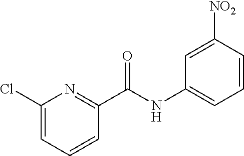 [SMILES: O=C(Nc1cccc([N+](=O)[O-])c1)c1cccc(Cl)n1]

HPLC-MS: Rt=2.68 min, (M+H)⁺ m/z 278, 279.

Yield: 52%

### Intermediate 21

### 3-Chloro-5-fluoro-N-(3-nitrophenyl)benzamide

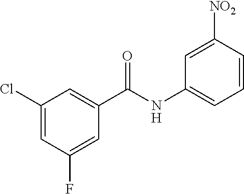 [SMILES: O=C(Nc1cccc([N+](=O)[O-])c1)c1cc(F)cc(Cl)c1]

HPLC-MS: Rt=3.27 min, (M+H)⁺ m/z 295, 297.

Yield: 60%

### Intermediate 22

### 6-Chloro-5-fluoro-N-(3-nitrophenyl)picolinamide

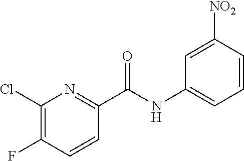 [SMILES: O=C(Nc1cccc([N+](=O)[O-])c1)c1ccc(F)c(Cl)n1]

HPLC-MS: Rt=3.18 min, [M+H]⁺ m/z 296, 298.

Yield: 66%.

### Intermediate 23

### 5-Chloro-6-fluoro-N-(3-nitrophenyl)nicotinamide

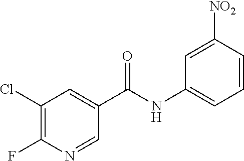 [SMILES: O=C(Nc1cccc([N+](=O)[O-])c1)c1cnc(F)c(Cl)c1]

HPLC-MS: Rt=3.09 min, [M+H]⁺ m/z 296, 298.

Yield: 27%.

### Intermediate 24

### N-(3-nitrophenyl)-3-(trifluoromethoxy)benzamide

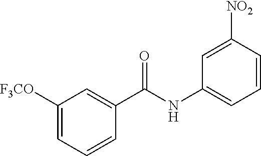 [SMILES: O=C(Nc1cccc([N+](=O)[O-])c1)c1cccc(OC(F)(F)F)c1]

HPLC-MS: Rt=3.32 min, [M+H]⁺ m/z 327.

Yield: 69%.

### Intermediate 25

### 3-Chloro-4-methyl-N-(3-nitrophenyl)benzamide

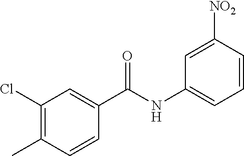 [SMILES: Cc1ccc(C(=O)Nc2cccc([N+](=O)[O-])c2)cc1Cl]

HPLC-MS: Rt=3.38 min, [M+H]⁺ m/z 291, 293.

Yield: 70%.

### Intermediate 26

### 3,4-Dichloro-N-(3-nitrophenyl)benzamide

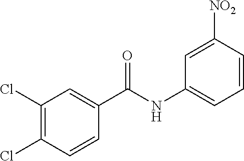 [SMILES: O=C(Nc1cccc([N+](=O)[O-])c1)c1ccc(Cl)c(Cl)c1]

HPLC-MS: Rt=3.49 min, [M+H]⁺ m/z 352.

Yield: 70%.

### Example 2

### N-(2-aminopyrimidin-4-yl)-3-chloro-4-methoxybenzamide

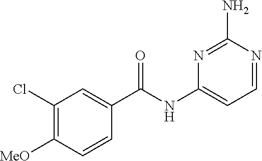 [SMILES: COc1ccc(C(=O)Nc2ccnc(N)n2)cc1Cl]

HPLC-MS: Rt=1.78 min, (M+H)⁺ m/z 279, 281.

¹H NMR (400 MHz, DMSO-d₆) δ 10.55 (s, 1H), 8.16 (d, J=5.6 Hz, 1H), 8.08 (d, J=2.2 Hz, 1H), 7.99 (dd, J=8.7, 2.3 Hz, 1H), 7.32 (d, J=5.6 Hz, 1H), 7.23 (d, J=8.8 Hz, 1H), 6.36 (s, 2H), 3.92 (s, 3H).

Yield: 11%

N-(4-aminopyrimidin-2-yl)-3-chloro-4-methoxybenzamide was also obtained in a 9% yield.

Structure determined by NOESY.

### Example 3

### N-(2-aminopyrimidin-4-yl)-3-chloro-4-fluorobenzamide

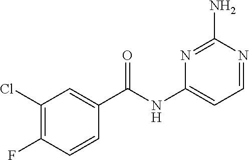 [SMILES: Nc1nccc(NC(=O)c2ccc(F)c(Cl)c2)n1]

HPLC-MS: Rt=1.80 min, (M+H)⁺ m/z 267, 267.

¹H NMR (400 MHz, DMSO-d₆) δ 10.76 (s, 1H), 8.21 (m, 2H), 7.99 (ddd, J=8.6, 4.7, 2.3 Hz, 1H), 7.55 (t, J=8.9 Hz, 1H), 7.32 (d, J=5.6 Hz, 1H), 6.41 (s, 2H).

Yield: 12%

N-(4-aminopyrimidin-2-yl)-3-chloro-4-fluorobenzamide (not according to the invention) was also obtained in as a by-product in a 16% yield.

Structure determined by NOESY.

### Example 4

### N-(2-aminopyrimidin-4-yl)-3-(trifluoromethoxy)benzamide

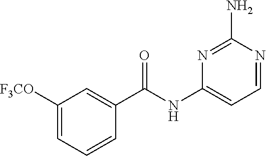 [SMILES: Nc1nccc(NC(=O)c2cccc(OC(F)(F)F)c2)n1]

HPLC-MS: Rt=2.03 min, (M+H)⁺ m/z 299, 300.

¹H NMR (400 MHz, DMSO-d₆) δ 10.83 (s, 1H), 8.21 (d, J=5.6 Hz, 1H), 8.02 (dt, J=7.4, 1.5 Hz, 1H), 7.94 (s, 1H), 7.72-7.53 (m, 2H), 7.35 (d, J=5.6 Hz, 1H), 6.42 (s, 2H).

Yield: 18%.

### Example 5

### N-(3-amino-5-chlorophenyl)-3-(trifluoromethoxy)benzamide

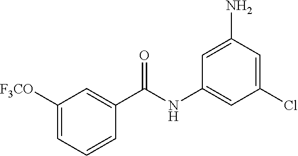 [SMILES: Nc1cc(Cl)cc(NC(=O)c2cccc(OC(F)(F)F)c2)c1]

HPLC-MS: Rt=3.15 min, (M+H)⁺ m/z 331, 332.

¹H NMR (400 MHz, CD₃OD) δ 7.91 (d, J=7.7 Hz, 1H), 7.81 (s, 1H), 7.61 (t, J=8.0 Hz, 1H), 7.50 (d, J=8.2 Hz, 1H), 7.02 (dd, J=4.6, 1.7 Hz, 2H), 6.51 (t, J=1.7 Hz, 1H).

Yield: 39%.

### Example 6

### N-(2-amino-6-methylpyrimidin-4-yl)-3-chlorobenzamide

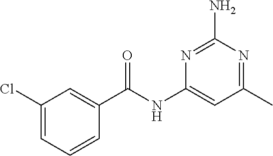 [SMILES: Cc1cc(NC(=O)c2cccc(Cl)c2)nc(N)n1]

HPLC-MS: Rt=1.83 min, (M+H)⁺ m/z 263, 265.

¹H NMR (400 MHz, DMSO-d₆) δ 10.65 (s, 1H), 8.01 (t, J=1.9 Hz, 1H), 7.96-7.87 (m, 1H), 7.66 (ddd, J=8.0, 2.1, 1.0 Hz, 1H), 7.53 (t, J=7.9 Hz, 1H), 7.27 (s, 1H), 6.33 (s, 2H), 2.25 (s, 3H).

Yield: 11%.

### General Procedure III:

In a particular embodiment according to synthetic routes A or C explained under the section “synthesis of compounds of formula (I)”, a compound of formula (IIIc) and a compound of formula (IIc) are reacted to prepared compounds of formula (VIc):

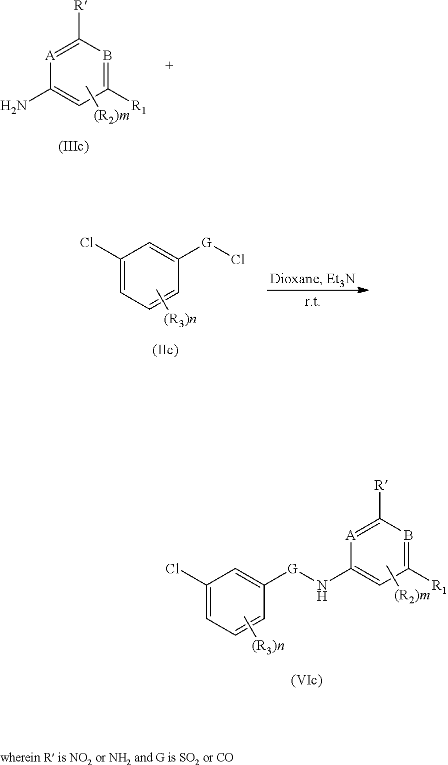

It must be noted that when R′ is an amino group the compounds of formula (VIb) are compounds of formula (I) according to the invention.

A solution of the appropriate compound of formula (IIIc) (1 eq) (ex: 2,6-diaminopyridine) and triethylamine (1 eq) in dry dioxane (4.5 mL/mmol) was prepared under inert atmosphere. Another solution of the appropriate acid or sulphonyl chloride of formula (IIc) (1 eq) (ex: 3-chlorobenzoyl chloride) in dry dioxane was prepared. Both solutions were added simultaneously dropwise at room temperature into a 50 mL flask (or the sulphonyl chloride added to the amine solution). After a period of 30 minutes stirring at room temperature (in some cases 60° C.), the reaction was completed. The solvent was removed under vacuum. The dry residue was taken up into ethyl acetate and washed with water. After extraction with ethyl acetate (3×), the combined organic layers were dried (magnesium sulphate), filtered and concentrated to yield the desired product (ex: N-(6-aminopyridin-2-yl)-3-chlorobenzamide).

### Example 7

### N-(6-aminopyridin-2-yl)-3-chlorobenzamide

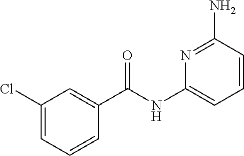 [SMILES: Nc1cccc(NC(=O)c2cccc(Cl)c2)n1]

HPLC-MS: Rt=1.82 min, (M+H)⁺ m/z 248, 250.

¹H NMR (400 MHz, MeOD) δ 9.51 (t, J=1.9 Hz, 1H), 9.42 (m, 1H), 9.15 (ddd, J=8.0, 2.1, 1.1 Hz, 1H), 9.10-8.98 (m, 2H), 8.95 (dd, J=7.8, 0.8 Hz, 1H), 7.90 (dd, J=8.0, 0.8 Hz, 1H).

Yield: 54%

### Example 8

### N-(2-aminopyrimidin-4-yl)-3-chlorobenzamide

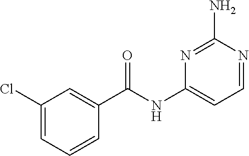 [SMILES: Nc1nccc(NC(=O)c2cccc(Cl)c2)n1]

HPLC-MS: Rt=1.73 min, (M+H)⁺ m/z 249, 251.

¹H NMR (400 MHz, DMSO-d₆) δ 10.74 (s, 1H), 8.20 (d, J=5.6 Hz, 1H), 8.02 (s, 1H), 7.92 (d, J=7.8 Hz, 1H), 7.67 (d, J=8.0 Hz, 1H), 7.53 (t, J=7.9 Hz, 1H), 7.34 (d, J=5.6 Hz, 1H), 6.42 (s, 2H, NH2).

Yield: 11%

Structure determined by NOESY.

N-(4-aminopyrimidin-2-yl)-3-chlorobenzamide (not according to the invention) was obtained as well in a 12% yield.

### Intermediate 27

### 3-Chloro-N-((3-chlorophenyl)sulfonyl)-N-(2-fluoro-4-methyl-5-nitrophenyl)benzenesulfonamide

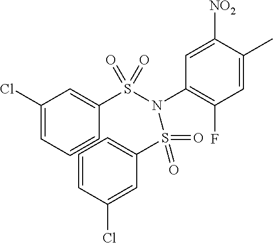 [SMILES: Cc1cc(F)c(N(S(=O)(=O)c2cccc(Cl)c2)S(=O)(=O)c2cccc(Cl)c2)cc1[N+](=O)[O-]]

HPLC-MS: Rt=3.62 min, [M+H]⁺ m/z 519, 521.

Yield: 79%

### Intermediate 28

### 3-Chloro-N-(2-fluoro-4-methyl-5-nitrophenyl)benzenesulfonamide

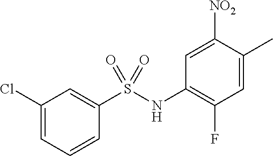 [SMILES: Cc1cc(F)c(NS(=O)(=O)c2cccc(Cl)c2)cc1[N+](=O)[O-]]

To a solution of 3-chloro-N-((3-chlorophenyl)sulfonyl)-N-(2-fluoro-4-methyl-5-nitrophenyl)benzenesulfonamide (295 mg, 0.568 mmol, 1 eq) in dry tetrahydrofuran (5 mL) under inert atmosphere was added tetrabutylammonium fluoride (TBAF) (0.624 mL, 0.624 mmol, 1M in tetrahydrofurane) dropwise at room temperature. After 3 h the reaction was complete. The solution was washed with water (5 mL) and extracted with ethyl acetate (3×). The combined organic extracts were dried (magnesium sulphate), filtered, and concentrated under vacuum. The crude was purified by column (hexane/ethyl acetate) to obtain 98% of desired product.

HPLC-MS: Rt=2.95 min, [M+H+H₂O]⁺ m/z 362, 364.

### General Procedure IV:

In a particular embodiment according to synthetic route D explained under the section “synthesis of compounds of formula (I)”, a compound of formula (IId) and a compound of formula (IId) are reacted to prepared compounds of formula (VId):

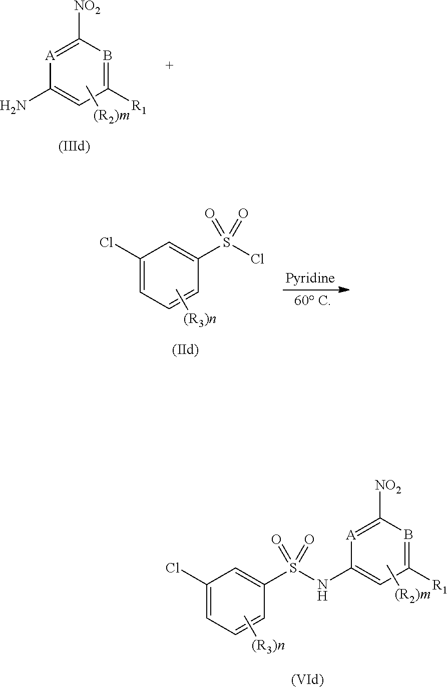

To a solution of the appropriate amine (ex: 2-fluoro-5-nitroaniline) (1 eq) in pyridine (1.9 mL/mmol), the appropriate sulfonyl chloride (1.2 eq) (ex: 3-chlorobenzenesulfonyl chloride) was added under inert atmosphere. The reaction mixture was stirred at 60° C. for 3 h. Pyridine was removed under reduced pressure, the residue was dissolved in ethyl acetate and HCl (1M) was added to the solution, and the mixture was extracted with ethyl acetate (3×). The residue was purified by flash column chromatography (hexanes/ethyl acetate) to obtain the desired amide (ex: 3-chloro-N-(2-fluoro-5-nitrophenyl)benzenesulfonamide).

### Intermediate 29

### 3-Chloro-N-(2-fluoro-5-nitrophenyl)benzenesulfonamide

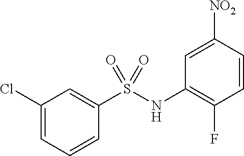 [SMILES: O=[N+]([O-])c1ccc(F)c(NS(=O)(=O)c2cccc(Cl)c2)c1]

HPLC-MS: Rt=2.88 min, [M+H]⁺ m/z 345, 347.

Yield: 73%

### Intermediate 30

### 3-Chloro-N-(3-nitrophenyl)benzenesulfonamide

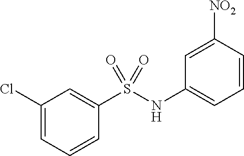 [SMILES: O=[N+]([O-])c1cccc(NS(=O)(=O)c2cccc(Cl)c2)c1]

HPLC-MS: Rt=2.82 min, [M+H+H₂O]⁺ m/z 330, 332.

Yield: 57%

### General Procedure V:

In a particular embodiment according to synthetic route B explained under the section “synthesis of compounds of formula (I)”, a compound of formula (IVe) is reacted to prepare a compound of formula (Ie):

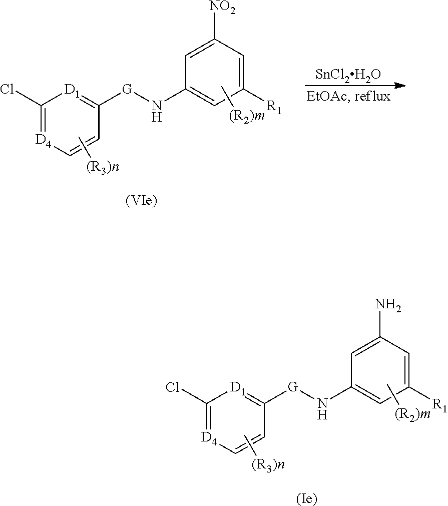

A mixture of the appropriate nitro intermediate (ex: 3-chloro-N-(4-methyl-3-nitrophenyl)benzamide) and SnCl₂.H₂O (4 eq) in ethyl acetate (12 mL/mmol) was refluxed overnight. On cooling, the reaction mixture was filtered through a short silica gel pad. The obtained solution was washed with sodium bicarbonate (sat. sol.) (5×) and extracted with ethyl acetate (3×). The combined organic layers were dried (magnesium sulphate), filtered and concentrated under vacuum. The residue was purified by flash column chromatography (dichloromethane/methanol) to yield the desired amino product (ex: N-(3-amino-4-methylphenyl)-3-chlorobenzamide).

### Example 9

### N-(3-amine-6-fluorophenyl)-3-chlorobenzamide

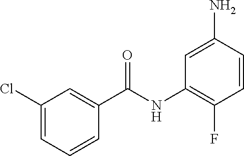 [SMILES: Nc1ccc(F)c(NC(=O)c2cccc(Cl)c2)c1]

HPLC-MS: Rt=2.10 min, (M+H)⁺ m/z 265, 267.

¹H NMR (400 MHz, DMSO-d₆) δ 10.37 (s, 1H), 7.99 (t, J=1.8 Hz, 1H), 7.91 (dd, J=7.8, 1.1 Hz, 1H), 7.74 (s, 1H), 7.67 (ddd, J=8.0, 2.1, 1.0 Hz, 1H), 7.57 (t, J=7.9 Hz, 1H), 7.31 (s, 1H), 7.18 (d, J=8.1 Hz, 1H).

Yield: 43%

### Example 10

### N-(3-amino-4-methylphenyl)-3-chlorobenzamide

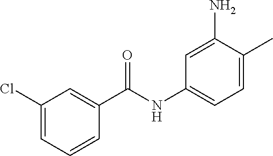 [SMILES: Cc1ccc(NC(=O)c2cccc(Cl)c2)cc1N]

HPLC-MS: Rt=2.30 min, (M+H)⁺ m/z 261, 263

¹H NMR (400 MHz, DMSO-d₆) δ 10.33 (s, 1H), 7.99 (t, J=1.9 Hz, 1H), 7.95-7.87 (m, 1H), 7.68-7.65 (m, 2H), 7.57 (t, J=8.0, 1H), 7.27 (d, J=7.6 Hz, 1H), 7.14 (d, J=8.2 Hz, 1H), 2.19 (s, 3H).

Yield: 35%

### Example 11

### N-(5-amino-2-fluoro-4-methylphenyl)-3-chlorobenzamide

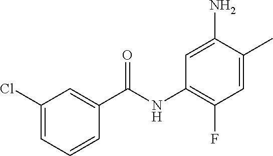 [SMILES: Cc1cc(F)c(NC(=O)c2cccc(Cl)c2)cc1N]

HPLC-MS: Rt=2.37 min, (M+H)⁺ m/z 279, 281.

¹H NMR (400 MHz, MeOD) δ 7.95 (t, J=1.8 Hz, 1H), 7.89-7.85 (m, 1H), 7.78 (d, J=6.9 Hz, 1H), 7.62 (ddd, J=8.0, 2.1, 1.0 Hz, 1H), 7.52 (t, J=7.9 Hz, 1H), 7.17 (d, J=11.0 Hz, 1H), 2.34 (s, 3H).

Yield: 43%

Synthetic Communications, 34(12), 2295-2301; 2004

### Example 12

### N-(3-aminophenyl)-3-bromobenzamide

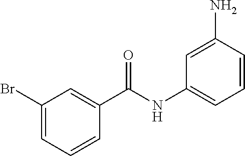 [SMILES: Nc1cccc(NC(=O)c2cccc(Br)c2)c1]

HPLC-MS: Rt=2.10 min, (M+H)⁺ m/z 291, 293.

¹H NMR (400 MHz, DMSO-d₆) δ 10.03 (s, 1H), 8.10 (s, 1H), 7.92 (d, J=7.8 Hz, 1H), 7.77 (m, 1H), 7.48 (t, J=7.9 Hz, 1H), 7.08 (s, 1H), 6.96 (t, J=7.9 Hz, 1H), 6.84 (d, J=8.9 Hz, 1H), 6.32 (dd, J=7.8, 1.2 Hz, 1H), 5.09 (s, 2H, NH2).

Yield: 64%

### Example 13

### N-(3-aminophenyl)-3,5-dichlorobenzamide

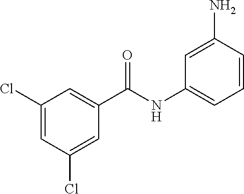 [SMILES: Nc1cccc(NC(=O)c2cc(Cl)cc(Cl)c2)c1]

HPLC-MS: Rt=2.47 min, (M+H)⁺ m/z 281, 283.

¹H NMR (400 MHz, DMSO-d₆) δ 10.11 (s, 1H), 7.94 (d, J=1.9 Hz, 2H), 7.84 (s, 1H), 7.07 (t, J=2.0 Hz, 1H), 6.97 (t, J=8.0 Hz, 1H), 6.84 (d, J=8.9 Hz, 1H), 6.33 (m, 1H), 5.12 (s, 2H, NH2).

Yield: 73%

### Example 14

### N-(3-aminophenyl)-5-chloro-2-(trifluoromethyl)benzamide

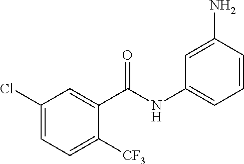 [SMILES: Nc1cccc(NC(=O)c2cc(Cl)ccc2C(F)(F)F)c1]

HPLC-MS: Rt=2.27 min, (M+H)⁺ m/z 315, 317.

¹H NMR (400 MHz, DMSO-d₆) δ 10.35 (s, 1H), 7.87 (d, J=8.5 Hz, 1H), 7.78 (ddd, J=7.6, 4.8, 4.0 Hz, 2H), 7.12 (s, 1H), 7.00 (t, J=8.1 Hz, 1H), 6.78 (d, J=7.8 Hz, 1H), 6.39 (d, J=7.5 Hz, 1H).

Yield: 60%

### Example 15

### N-(3-aminophenyl)-2,5-dichlorobenzamide

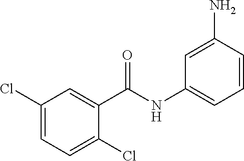 [SMILES: Nc1cccc(NC(=O)c2cc(Cl)ccc2Cl)c1]

HPLC-MS: Rt=2.15 min, (M+H)⁺ m/z 281, 283.

¹H NMR (400 MHz, DMSO-d₆) δ 10.25 (s, 1H), 7.66 (dd, J=2.2, 0.5 Hz, 1H), 7.57 (dd, J=3.6, 1.4 Hz, 2H), 7.07 (t, J=2.0 Hz, 1H), 6.96 (t, J=8.0 Hz, 1H), 6.76 (m, 1H), 6.33 (ddd, J=8.0, 2.2, 0.9 Hz, 1H), 5.20 (s, 2H, NH2).

Yield: 81%

### Example 16

### N-(3-aminophenyl)-5-chloro-2-fluorobenzamide

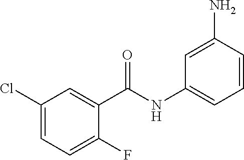 [SMILES: Nc1cccc(NC(=O)c2cc(Cl)ccc2F)c1]

HPLC-MS: Rt=2.08 min, (M+H)⁺ m/z 265, 267.

¹H NMR (400 MHz, DMSO-d₆) δ 10.19 (s, 1H), 7.68 (dd, J=5.9, 2.7 Hz, 1H), 7.62 (ddd, J=8.8, 4.4, 2.8 Hz, 1H), 7.40 (t, J=9.2 Hz, 1H), 7.04 (t, J=2.0 Hz, 1H), 6.96 (t, J=8.0 Hz, 1H), 6.78 (d, J=7.9 Hz, 1H), 6.32 (ddd, J=8.0, 2.2, 1.0 Hz, 1H), 5.12 (s, 2H).

Yield: 63%

### Example 17

### N-(3-aminophenyl)-5-chloro-2-methoxybenzamide

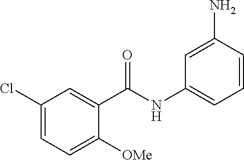 [SMILES: COc1ccc(Cl)cc1C(=O)Nc1cccc(N)c1]

HPLC-MS: Rt=2.27 min, (M+H)⁺ m/z 277, 279.

¹H NMR (400 MHz, DMSO-d₆) δ 9.87 (s, 1H), 7.56 (s, 1H), 7.52 (dd, J=8.8, 2.8 Hz, 1H), 7.19 (d, J=8.9 Hz, 1H), 7.05 (t, J=2.0 Hz, 1H), 6.93 (t, J=8.0 Hz, 1H), 6.76 (d, J=8.0 Hz, 1H), 6.29 (ddd, J=8.0, 2.2, 0.9 Hz, 1H), 5.08 (s, 2H), 3.87 (s, 3H).

Yield: 72%

### Example 18

### N-(3-aminophenyl)-3-chloro-4-fluorobenzamide

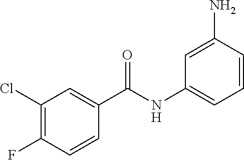 [SMILES: Nc1cccc(NC(=O)c2ccc(F)c(Cl)c2)c1]

HPLC-MS: Rt=2.13 min, (M+H)⁺ m/z 265, 267.

¹H NMR (400 MHz, DMSO-d₆) δ 10.04 (s, 1H), 8.16 (dd, J=7.2, 2.2 Hz, 1H), 7.96 (ddd, J=8.7, 4.8, 2.2 Hz, 1H), 7.57 (t, J=8 Hz, 1H), 7.07 (t, J=2.0 Hz, 1H), 6.97 (t, J=8.0 Hz, 1H), 6.84 (dd, J=7.5, 1.4 Hz, 1H), 6.33 (ddd, J=7.9, 2.2, 0.9 Hz, 1H), 5.10 (s, 2H).

Yield: 60%

### Example 19

### N-(3-aminophenyl)-3-chloro-2-methylbenzamide

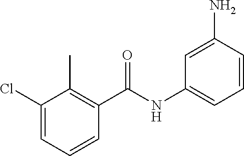 [SMILES: Cc1c(Cl)cccc1C(=O)Nc1cccc(N)c1]

HPLC-MS: Rt=2.10 min, (M+H)⁺ m/z 260, 262.

¹H NMR (400 MHz, DMSO-d₆) δ 10.10 (s, 1H), 7.54 (dd, J=7.7, 1.4 Hz, 1H), 7.35 (m, 2H), 7.08 (t, J=2.0 Hz, 1H), 6.94 (t, J=8.0 Hz, 1H), 6.77 (m, 1H), 6.31 (ddd, J=8.0, 2.1, 0.9 Hz, 1H), 5.09 (s, 2H), 2.36 (s, 3H).

Yield: 89%

### Example 20

### N-(3-aminophenyl)-3-chloro-4-methoxybenzamide

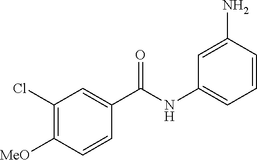 [SMILES: COc1ccc(C(=O)Nc2cccc(N)c2)cc1Cl]

HPLC-MS: Rt=2.00 min, (M+H)⁺ m/z 277, 279.

¹H NMR (400 MHz, DMSO-d₆) δ 9.88 (s, 1H), 8.05 (d, J=2.2 Hz, 1H), 7.95 (dd, J=8.6, 2.2 Hz, 1H), 7.27 (d, J=8.8 Hz, 1H), 7.08 (d, J=2.0 Hz, 1H), 6.96 (t, J=7.9 Hz, 1H), 6.85 (ddd, J=8.0, 1.9, 1.0 Hz, 1H), 6.31 (ddd, J=7.9, 2.2, 1.0 Hz, 1H), 5.07 (s, 2H), 3.94 (s, 3H).

Yield: 57%

### Example 21

### N-(3-aminophenyl)-3-chloro-5-methoxybenzamide

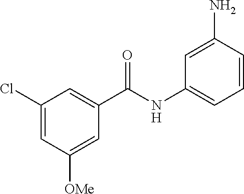 [SMILES: COc1cc(Cl)cc(C(=O)Nc2cccc(N)c2)c1]

HPLC-MS: Rt=2.18 min, (M+H)⁺ m/z 277, 279.

¹H NMR (400 MHz, DMSO-d₆) δ 9.99 (s, 1H), 7.54 (t, J=1.6 Hz, 1H), 7.43 (dd, J=2.3, 1.4 Hz, 1H), 7.23 (t, J=2 Hz, 1H), 7.06 (t, J=2.0 Hz, 1H), 6.96 (t, J=7.9 Hz, 1H), 6.84 (d, J=8.0 Hz, 1H), 6.33 (m, 1H), 5.09 (s, 2H), 3.86 (s, 3H).

Yield: 72%

### Example 22

### N-(5-amino-2-fluorophenyl)-3-chlorobenzenesulfonamide

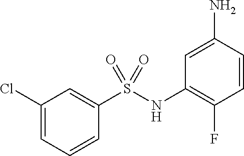 [SMILES: Nc1ccc(F)c(NS(=O)(=O)c2cccc(Cl)c2)c1]

HPLC-MS: Rt=2.23 min, (M+H)⁺ m/z 342, 344.

¹H NMR (400 MHz, MeOD) δ 7.75 (t, J=1.9 Hz, 1H), 7.72-7.64 (m, 1H), 7.63-7.55 (m, 2H), 7.47 (t, J=8.0 Hz, 1H), 7.23-7.15 (m, 1H), 7.12 (ddd, J=8.8, 4.1, 2.7 Hz, 1H).

Yield: 69%

### Example 23

### N-(5-amino-2-fluoro-4-methylphenyl)-3-chlorobenzenesulfonamide

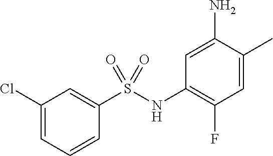 [SMILES: Cc1cc(F)c(NS(=O)(=O)c2cccc(Cl)c2)cc1N]

HPLC-MS: Rt=2.48 min, (M+H)⁺ m/z 356, 358.

¹H NMR (400 MHz, DMSO-d₆) δ 10.08 (s, 1H), 7.75-7.71 (m, 2H), 7.65 (dt, J=7.8, 1.4 Hz, 1H), 7.62-7.55 (m, 1H), 6.86 (d, J=11.0 Hz, 1H), 6.74 (d, J=7.1 Hz, 1H), 2.04 (s, 3H).

Yield: 49%

### Example 24

### N-(3-aminophenyl)-3-chlorobenzenesulfonamide

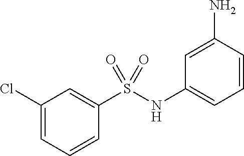 [SMILES: Nc1cccc(NS(=O)(=O)c2cccc(Cl)c2)c1]

HPLC-MS: Rt=2.27 min, (M+H)⁺ m/z 283, 285.

¹H NMR (400 MHz, DMSO-d₆) δ 10.06 (s, 1H), 7.74 (t, J=1.9 Hz, 1H), 7.69 (m, 2H), 7.59 (t, J=7.9 Hz, 1H), 6.86 (t, J=7.9 Hz, 1H), 6.40 (s, 1H), 6.26 (d, J=8.1 Hz, 2H).

Yield: 28%

### Example 25

### N-(3-aminophenyl)-2-chloroisonicotinamide

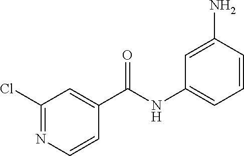 [SMILES: Nc1cccc(NC(=O)c2ccnc(Cl)c2)c1]

HPLC-MS: Rt=1.70 min, (M+H)⁺ m/z 248, 250.

¹H NMR (400 MHz, DMSO-d₆) δ 10.24 (s, 1H), 8.58 (dd, J=5.1, 0.7 Hz, 1H), 7.93 (dd, J=1.5, 0.7 Hz, 1H), 7.82 (dd, J=5.1, 1.5 Hz, 1H), 7.05 (t, J=2.0 Hz, 1H), 6.96 (t, J=8.0 Hz, 1H), 6.83 (ddd, J=8.1, 2.0, 1.0 Hz, 1H), 6.33 (ddd, J=8.0, 2.2, 1.0 Hz, 1H), 5.13 (s, 2H).

Yield: 67%

### Example 26

### N-(3-aminophenyl)-6-chloropicolinamide

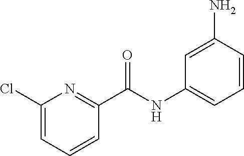 [SMILES: Nc1cccc(NC(=O)c2cccc(Cl)n2)c1]

HPLC-MS: Rt=2.10 min, (M+H)⁺ m/z 248, 250.

¹H NMR (400 MHz, DMSO-d₆) δ 9.99 (s, 1H), 8.08 (m, 2H), 7.76 (dd, J=6.9, 1.9 Hz, 1H), 7.13 (t, J=2.1 Hz, 1H), 6.97 (t, J=7.9 Hz, 1H), 6.90 (dt, J=8.1, 1.4 Hz, 1H), 6.39-6.27 (m, 1H), 5.10 (s, 2H).

Yield: 65%

### Example 27

### N-(3-aminophenyl)-3-chloro-5-fluorobenzamide

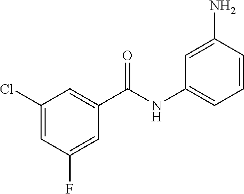 [SMILES: Nc1cccc(NC(=O)c2cc(F)cc(Cl)c2)c1]

HPLC-MS: Rt=2.32 min, (M+H)⁺ m/z 265, 267.

¹H NMR (400 MHz, DMSO-d₆) δ 10.06 (s, 1H), 7.83 (t, J=1.6 Hz, 1H), 7.69 (dddd, J=23.2, 8.6, 2.5, 1.7 Hz, 2H), 7.05 (t, J=2.1 Hz, 1H), 6.95 (t, J=7.9 Hz, 1H), 6.83 (ddd, J=8.0, 2.0, 1.0 Hz, 1H), 6.32 (ddd, J=8.0, 2.3, 1.0 Hz, 1H), 5.10 (s, 2H).

Yield: 75%

### Example 28

### N-(3-aminophenyl)-6-chloro-5-fluoropicolinamide

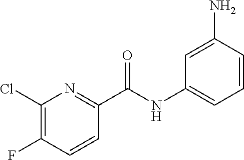 [SMILES: Nc1cccc(NC(=O)c2ccc(F)c(Cl)n2)c1]

HPLC-MS: Rt=2.25 min, (M+H)⁺ m/z 266, 268.

¹H NMR (400 MHz, DMSO-d₆) δ 10.04 (s, 1H), 8.16 (dd, J=6.0, 2.8 Hz, 2H), 7.15 (t, J=2.0 Hz, 1H), 6.99 (t, J=7.9 Hz, 1H), 6.94-6.87 (m, 1H), 6.35 (ddd, J=7.9, 2.1, 1.0 Hz, 1H), 5.12 (s, 2H).

Yield: 74%.

### Example 29

### N-(3-aminophenyl)-5-chloro-6-fluoronicotinamide

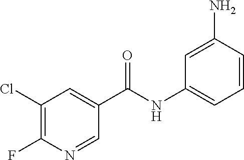 [SMILES: Nc1cccc(NC(=O)c2cnc(F)c(Cl)c2)c1]

HPLC-MS: Rt=2.02 min, (M+H)⁺ m/z 266, 268.

¹H NMR (400 MHz, DMSO-d₆) δ 10.20 (s, 1H), 8.68 (ddd, J=11.0, 5.5, 1.7 Hz, 2H), 7.06 (s, 1H), 6.99 (t, J=8.0 Hz, 1H), 6.90-6.80 (m, 1H), 6.35 (ddd, J=8.0, 2.2, 0.9 Hz, 1H), 5.18 (s, 2H).

Yield: 70%.

### Example 30

### N-(3-aminophenyl)-3-(trifluoromethoxy)benzamide

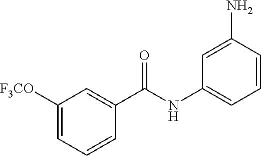 [SMILES: Nc1cccc(NC(=O)c2cccc(OC(F)(F)F)c2)c1]

HPLC-MS: Rt=2.48 min, (M+H)⁺ m/z 298, 298.

¹H NMR (400 MHz, DMSO-d₆) δ 10.07 (s, 1H), 8.00-7.95 (m, 1H), 7.87 (s, 1H), 7.66 (t, J=8.0 Hz, 1H), 7.58 (ddt, J=8.2, 2.3, 1.0 Hz, 1H), 7.07 (t, J=2.0 Hz, 1H), 6.97 (t, J=7.9 Hz, 1H), 6.85 (ddd, J=8.0, 1.9, 1.0 Hz, 1H), 6.33 (ddd, J=7.9, 2.2, 1.0 Hz, 1H), 5.10 (s, 2H).

Yield: 70%.

### Example 31

### N-(3-aminophenyl)-3-chloro-4-methylbenzamide

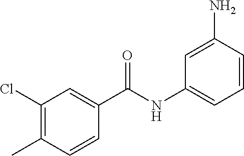 [SMILES: Cc1ccc(C(=O)Nc2cccc(N)c2)cc1Cl]

HPLC-MS: Rt=2.40 min, (M+H)⁺ m/z 261, 263.

¹H NMR (400 MHz, DMSO-d₆) δ 9.97 (s, 1H), 7.99 (d, J=1.8 Hz, 1H), 7.82 (dd, J=7.9, 1.8 Hz, 1H), 7.50 (d, J=7.9 Hz, 1H), 7.09 (s, 1H), 6.97 (t, J=7.9 Hz, 1H), 6.86 (ddd, J=8.0, 1.9, 1.0 Hz, 1H), 6.33 (ddd, J=7.9, 2.2, 1.0 Hz, 1H), 5.09 (s, 2H), 2.41 (s, 3H).

Yield: 63%.

### Example 32

### N-(3-amino-5-methylphenyl)-3-chlorobenzamide

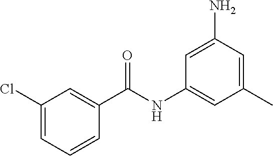 [SMILES: Cc1cc(N)cc(NC(=O)c2cccc(Cl)c2)c1]

HPLC-MS: Rt=2.33 min, (M+H)⁺ m/z 261, 263.

¹H NMR (400 MHz, CD₃OD) δ 7.91 (t, J=1.8 Hz, 1H), 7.86-7.79 (m, 1H), 7.57 (ddd, J=8.0, 2.1, 1.1 Hz, 1H), 7.49 (t, J=7.8 Hz, 1H), 6.96 (t, J=1.7 Hz, 1H), 6.81 (s, 1H), 6.51-6.32 (m, 1H), 2.24 (s, 3H).

Yield: 67%

### Example 33

### N-(6-aminopyridin-2-yl)-3-chlorobenzamide

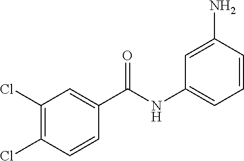 [SMILES: Nc1cccc(NC(=O)c2ccc(Cl)c(Cl)c2)c1]

HPLC-MS: Rt=2.57 min, (M+H)⁺ m/z 281, 283.

¹H NMR (400 MHz, DMSO-d₆) δ 10.09 (s, 1H), 8.18 (d, J=2.0 Hz, 1H), 7.91 (dd, J=8.4, 2.1 Hz, 1H), 7.80 (d, J=8.4 Hz, 1H), 7.08 (d, J=2.0 Hz, 1H), 6.97 (t, J=8.0 Hz, 1H), 6.84 (dd, J=4.9, 4.1 Hz, 1H), 6.33 (ddd, J=8.0, 2.1, 0.9 Hz, 1H), 5.11 (s, 2H).

Yield: 76%

### General Procedure VI:

In a particular embodiment according to synthetic route E explained under the section “synthesis of compounds of formula (I)”, a compound of formula (IIf) is reacted to prepare compounds of formula (If):

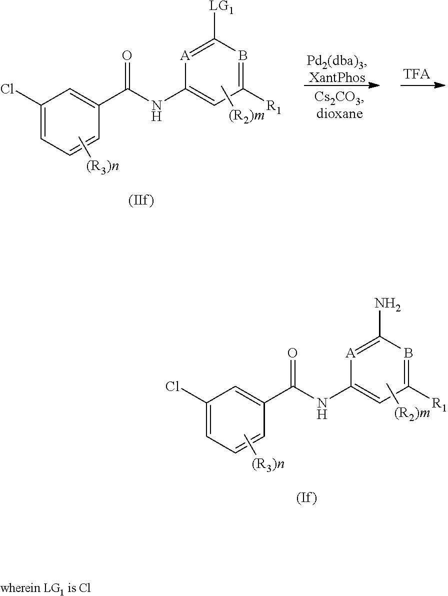

A mixture of the appropriate chloride (ex: 3-chloro-N-(2-chloropyridin-4-yl)benzamide) (1 eq), carbamic acid isopropyl ester (1.5 eq), Pd₂(dba)₃ (0.1 eq), XantPhos (0.2 eq) and cesium carbonate (2 eq) in dioxane (5.3 mL/mmol) was heated at 140° C. for 2-5 h under nitrogen atmosphere. The mixture was filtered through a celite pad and the filtrate concentrated under reduced pressure. The residue was purified by silica gel flash column chromatography (dichloromethane/methanol) to obtain the desired intermediate compound (ex: tert-butyl (4-(3-chlorobenzamido)pyridin-2-yl)carbamate).

Trifluoroacetic acid (TFA) in dichloromethane (1:1) was added to a solution of the BOC-intermediate (ex: tert-butyl (4-(3-chlorobenzamido)pyridin-2-yl)carbamate) (1 eq) in dichloromethane (4.3 mL/mmol). The solution was stirred at room temperature for 3-5 h and concentrated under vacuum. The crude was dissolved in ethyl acetate, the obtained solution was washed with sodium bicarbonate (sat. sol.) (1×) and extracted with ethyl acetate (1×). The combined organic layers were dried (magnesium sulphate), filtered and concentrated under vacuum. The resultant residue was purified by silica gel flash column chromatography (dichloromethane/methanol) to obtain the free amine compound (ex: N-(2-aminopyridin-4-yl)-3-chlorobenzamide).

### Example 34

### N-(2-aminopyridin-4-yl)-3-chlorobenzamide

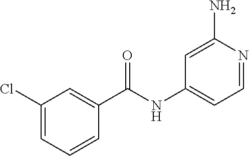 [SMILES: Nc1cc(NC(=O)c2cccc(Cl)c2)ccn1]

Trifluoroacetic acid (TFA) (0.2 mL) was added to a solution of tert-butyl (2-(3-chlorobenzamido)pyridin-4-yl)carbamate (80 mg, 0.23 mmol) in dichloromethane (1 mL). The solution was stirred at room temperature for 5 h and concentrated under vacuum. The resultant residue was purified by silica gel flash column chromatography (dichloromethane/methanol) to obtain 22 mg (24%, two steps) of N-(2-aminopyridin-4-yl)-3-chlorobenzamide as a white solid.

HPLC-MS: Rt=2.28 min, (M+H)⁺ m/z 248, 250.

¹H NMR (400 MHz, DMSO-d₆) δ 11.01 (s, 1H, CONH), 8.01 (t, J=1.8 Hz, 1H), 7.91 (m, 4H), 7.74 (ddd, J=8.0, 2.1, 1.0 Hz, 1H), 7.70 (d, J=1.8 Hz, 1H), 7.62 (t, J=7.9 Hz, 1H), 7.03 (dd, J=7.2, 2.1 Hz, 1H).+

### Example 35

### N-(2-amino-6-(pyridin-2-yloxy)pyridin-4-yl)-3-chlorobenzamide

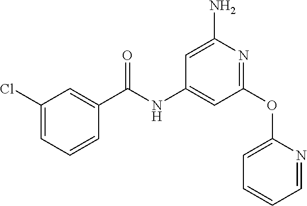 [SMILES: Nc1cc(NC(=O)c2cccc(Cl)c2)cc(Oc2ccccn2)n1]

HPLC-MS: Rt=2.32 min, (M+H)⁺ m/z 341, 343.

¹H NMR (400 MHz, CD₃OD) δ 8.42 (d, J=2.7 Hz, 1H), 8.38 (dd, J=4.8, 1.4 Hz, 1H), 7.93-7.91 (m, 1H), 7.83 (ddd, J=7.7, 1.7, 1.1 Hz, 1H), 7.68 (ddd, J=8.4, 2.7, 1.4 Hz, 1H), 7.60 (ddd, J=8.0, 2.1, 1.1 Hz, 1H), 7.55-7.47 (m, 2H), 6.92 (d, J=1.5 Hz, 1H), 6.54 (d, J=1.5 Hz, 1H).

Yield: 42%.

### Example 36

### N-(2-amino-6-chloropyridin-4-yl)-3-chlorobenzamide

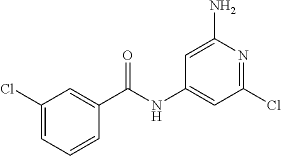 [SMILES: Nc1cc(NC(=O)c2cccc(Cl)c2)cc(Cl)n1]

HPLC-MS: Rt=2.73 min, (M+H)⁺ m/z 282, 284.

¹H NMR (400 MHz, CD₃OD) δ 7.95 (s, 1H), 7.86 (d, J=7.8 Hz, 1H), 7.64 (d, J=8.2 Hz, 1H), 7.53 (t, J=8.0 Hz, 1H), 7.29 (s, 1H), 7.02 (s, 1H).

Yield: 9%.

### Example 37

### N-(2-aminopyridin-4-yl)-3-(trifluoromethoxy)benzamide

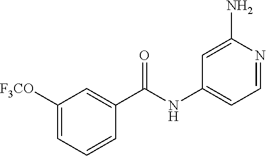 [SMILES: Nc1cc(NC(=O)c2cccc(OC(F)(F)F)c2)ccn1]

HPLC-MS: Rt=1.80 min, [M+H]⁺ m/z, 298, 300.

¹H NMR (400 MHz, CDCl₃) δ 7.86 (d, J=7.8 Hz, 1H), 7.82-7.71 (m, 3H), 7.69 (d, J=2.1 Hz, 1H), 7.46 (td, J=8.0, 3.4 Hz, 2H), 7.38-7.30 (m, 2H), 6.30 (dd, J=5.9, 2.2 Hz, 1H).

Yield: 9% (two steps)

### General Procedure VII:

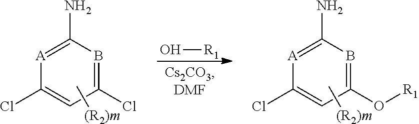

A 10 mL round-bottomed flask was charged with the appropriate aromatic chloride derivative (ex: 4,6-dichloropyrimidin-2-amine) (1 eq), the appropriate alcohol (ex: 3-hydroxypyridine) (1 eq), cesium carbonate (3 eq), and dimethylformamide (1.6 mL/mmol). The reaction mixture was heated at 100° C. for 1-2 h (if heated conventionally); or at 900 W for 2 minutes (if irradiated in microwave). After consumption of the started materials (as observed by thin layer chromatography), reaction mixture was cooled to room temperature; volatiles were removed under vacuum and crude extracted was partitioned between water and ethyl acetate. The organic phase was separated, washed successively with water (3×), dried over sodium sulphate, filtered and concentrated under vacuum to provide the wanted ether intermediate (ex: 4-chloro-6-(pyridin-3-yloxy)pyrimidin-2-amine). In some cases, the intermediate was purified by flash column chromatography (ethyl acetate/hexanes) or in some cases, taken further to next step without any purification.

### Intermediate 31

### 4-Chloro-6-(pyridin-2-yloxy)pyrimidin-2-amine

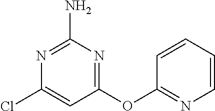 [SMILES: Nc1nc(Cl)cc(Oc2ccccn2)n1]

HPLC-MS: Rt=1.95 min, (M+H)⁺ m/z 223, 225.

Yield: 85%.

### Intermediate 32

### 4-Chloro-6-phenoxypyrimidin-2-amine

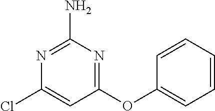 [SMILES: Nc1nc(Cl)cc(Oc2ccccc2)n1]

HPLC-MS: Rt=2.75 min, (M+H)⁺ m/z 222, 224.

Yield: 44%.

### Intermediate 33

### 2-Chloro-6-(pyridin-2-yloxy)pyridin-4-amine

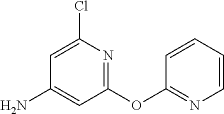 [SMILES: Nc1cc(Cl)nc(Oc2ccccn2)c1]

HPLC-MS: Rt=1.76 min, (M+H)⁺ m/z 222, 224.

Yield: 26%.

### Intermediate 34

### 4-Chloro-6-(3-chlorophenoxy)pyrimidin-2-amine

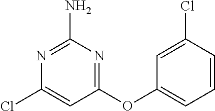 [SMILES: Nc1nc(Cl)cc(Oc2cccc(Cl)c2)n1]

HPLC-MS: (M+H)⁺ m/z 255.8.

### Intermediate 35

### 4-Chloro-6-(quinolin-3-yloxy)pyrimidin-2-amine

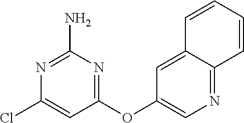 [SMILES: Nc1nc(Cl)cc(Oc2cnc3ccccc3c2)n1]

HPLC-MS: Rt=(M+H)⁺ m/z 273.0.

### Intermediate 36

### 4-Chloro-6-(quinolin-5-yloxy)pyrimidin-2-amine

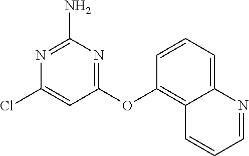 [SMILES: Nc1nc(Cl)cc(Oc2cccc3ncccc23)n1]

HPLC-MS: Rt=(M+H)⁺ m/z 273.0.

### Intermediate 37

### 4-Chloro-6-(pyridin-4-yloxy)pyrimidin-2-amine

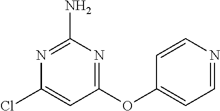 [SMILES: Nc1nc(Cl)cc(Oc2ccncc2)n1]

HPLC-MS: Rt=(M+H)⁺ m/z 223.0.

### General Procedure VIII

In a 25 mL round-bottomed the appropriate hydroxyl compound (ex: benzyl alcohol) (1.1 eq) and NaH (1.1-2.5 eq) were stirred in tetrahydrofuran (1.6 mL/mmol) at 0° C. for 30 minutes or at room temperature for 10 minutes. A solution of 4,6-dichloropyrimidin-2-amine (1 eq) in dimethylformamide (0.6 mL/mmol) was added. The reaction mixture was stirred at room temperature for 1 h or at 80° C. until consumption of the starting materials. The reaction was cooled and water was added, followed by extraction with dichloromethane. The combined organic phases were separated, washed successively with water (3×5 mL), dried over sodium sulphate, filtered and concentrated under vacuum. The crude was either purified by flash column chromatography (ethyl acetate/Hexanes) or taken further to next step without any purification.

### Intermediate 38

### 4-(Benzyloxy)-6-chloropyrimidin-2-amine

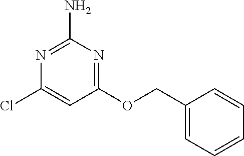 [SMILES: Nc1nc(Cl)cc(OCc2ccccc2)n1]

HPLC-MS: Rt=2.97 min, (M+H)⁺ m/z 236, 238.

Yield: 98%.

### Intermediate 39

### 4-Chloro-6-((tetrahydro-2H-pyran-4-yl)oxy)pyrimidin-2-amine

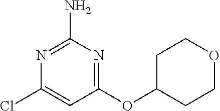 [SMILES: Nc1nc(Cl)cc(OC2CCOCC2)n1]

HPLC-MS: Rt=(M+H)⁺ m/z 229.9.

### Intermediate 40

### tert-Butyl (4-((2-amino-6-chloropyrimidin-4-yl)oxy)cyclohexyl)carbamate

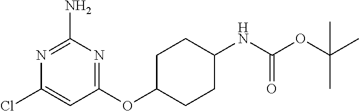 [SMILES: CC(C)(C)OC(=O)NC1CCC(Oc2cc(Cl)nc(N)n2)CC1]

HPLC-MS: Rt=(M+H)⁺ m/z 342.9.

### Intermediate 41

### 4-Chloro-6-((1-methylpiperidin-4-yl)oxy)pyrimidin-2-amine

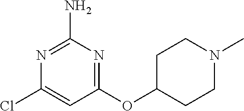 [SMILES: CN1CCC(Oc2cc(Cl)nc(N)n2)CC1]

HPLC-MS: Rt=(M+H)⁺ m/z 242.9.

### Intermediate 42

### 4-Chloro-6-methoxypyrimidin-2-amine

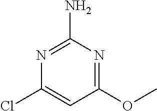

HPLC-MS: Rt=(M+H)⁺ m/z 159.9.

### General Procedure IX

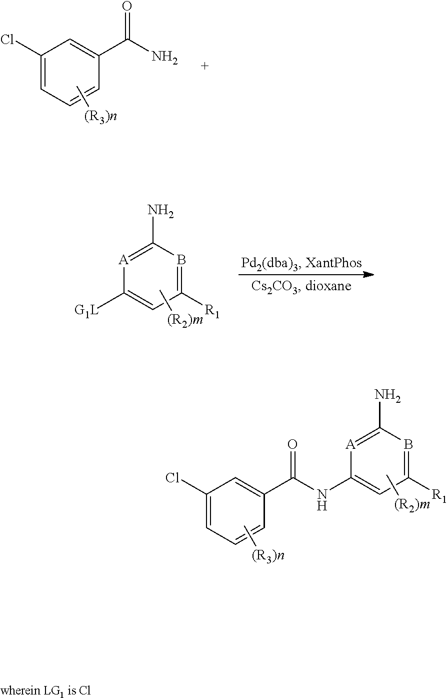

A mixture of the appropriate chloride (ex: 4-chloro-6-(pyridin-2-yloxy)pyrimidin-2-amine) (1 eq), the appropriate amide (ex: 3-chlorobenzamide) (1.5 eq), Pd₂(dba)₃ (0.1 eq.), XantPhos (0.2 eq) and cesium carbonate (2 eq) in dioxane (5.3 mL/mmol) was heated at 140° C. for 0.5-2 h under nitrogen atmosphere. The mixture was filtered through a celite pad and the filtrate concentrated under reduced pressure. The residue was purified by flash column chromatography (dichloromethane/methanol) or preparative HPLC to afford desired products (ex: N-(2-amino-6-(pyridin-2-yloxy)pyrimidin-4-yl)-3-chlorobenzamide).

### Example 38

N-(2-amino-6-(pyridin-3-yloxy)pyrimidin-4-yl)-3-chlorobenzamide

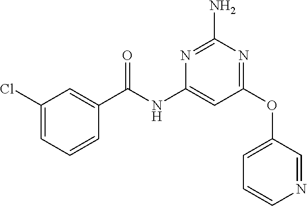 [SMILES: Nc1nc(NC(=O)c2cccc(Cl)c2)cc(Oc2cccnc2)n1]

HPLC-MS: Rt=2.55 min, (M+H)⁺ m/z 342, 344.

¹H NMR (400 MHz, CDCl₃) δ 9.29 (s, 1H), 8.59 (d, J=2.6 Hz, 1H), 8.54 (dd, J=4.8, 1.3 Hz, 1H), 7.97 (t, J=1.8 Hz, 1H), 7.88 (d, J=7.9 Hz, 1H), 7.63 (ddd, J=8.4, 2.6, 1.4 Hz, 1H), 7.58 (ddd, J=8.0, 2.0, 0.9 Hz, 1H), 7.51-7.45 (m, 2H), 7.40 (s, 1H), 5.39 (s, 2H).

Yield: 30%.

### Example 39

### N-(2-amino-6-phenoxypyrimidin-4-yl)-3-chlorobenzamide

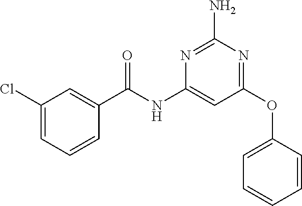 [SMILES: Nc1nc(NC(=O)c2cccc(Cl)c2)cc(Oc2ccccc2)n1]

HPLC-MS: Rt=3.10 min, (M+H)⁺ m/z 341, 343.

¹H NMR (400 MHz, CD₃OD) δ 7.93 (dd, J=2.7, 1.0 Hz, 1H), 7.85 (ddd, J=7.7, 1.7, 1.1 Hz, 1H), 7.62 (ddd, J=8.1, 2.1, 1.1 Hz, 1H), 7.51 (t, J=7.9 Hz, 1H), 7.45 (dd, J=8.3, 7.6 Hz, 2H), 7.33-7.23 (m, 1H), 7.19-7.11 (m, 2H), 6.98 (s, 1H).

Yield: 14%

### Example 40

### N-(2-amino-6-(benzyloxy)pyrimidin-4-yl)-3-chlorobenzamide

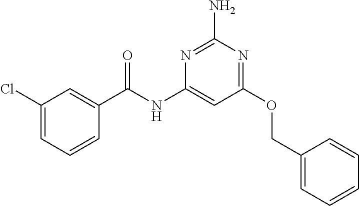 [SMILES: Nc1nc(NC(=O)c2cccc(Cl)c2)cc(OCc2ccccc2)n1]

HPLC-MS: Rt=3.22 min, (M+H)⁺ m/z 355, 357.

¹H NMR (400 MHz, CD₃OD) δ 7.95 (t, J=1.8 Hz, 1H), 7.86 (d, J=7.8 Hz, 1H), 7.62 (ddd, J=8.0, 2.0, 1.0 Hz, 1H), 7.52 (t, J=7.9 Hz, 1H), 7.44 (d, J=7.2 Hz, 2H), 7.37 (dd, J=8.1, 6.5 Hz, 2H), 7.32 (d, J=7.2 Hz, 1H), 6.97 (s, 1H), 5.36 (s, 2H).

Yield: 16%.

### Example 41

### N-[2-Amino-6-(3-chloro-phenoxy)-pyrimidin-4-yl]-3-chloro-benzamide

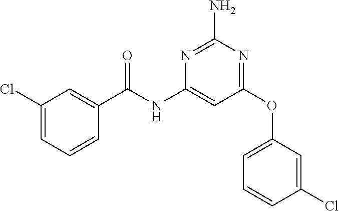 [SMILES: Nc1nc(NC(=O)c2cccc(Cl)c2)cc(Oc2cccc(Cl)c2)n1]

ES-MS (M+H)⁺ m/z 376.

¹H NMR (400 MHz, DMSO-d₆): δ 10.82 (s, 1H), 8.01 (s, 1H), 7.91 (d, J=7.6 Hz, 1H), 7.66 (d, J=8.0 Hz, 1H), 7.53 (dd, J=7.2, 8.0 Hz, 1H), 7.47 (t, J=8.0 Hz, 1H), 7.30-7.38 (m, 2H), 7.19 (d, J=8.0 Hz, 1H), 6.96 (s, 1H), 6.54 (br s, —NH₂).

Yield: 12% over 2 steps.

### Example 42

### N-[2-Amino-6-(quinolin-3-yloxy)-pyrimidin-4-yl]-3-chloro-benzamide

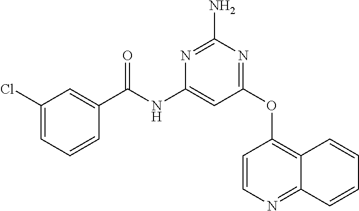 [SMILES: Nc1nc(NC(=O)c2cccc(Cl)c2)cc(Oc2ccnc3ccccc23)n1]

ES-MS (M+H)⁺ m/z 394.0.

¹H NMR (400 MHz, DMSO-d6): δ 10.90 (s, 1H), 8.84 (s, 1H), 8.25 (s, 1H), 7.97-8.08 (m, 3H), 7.92 (d, J=7.6 Hz, 1H), 7.77 (t, J=7.6 Hz, 1H), 7.67 (m, 2H), 7.54 (t, J=7.6 Hz, 1H), 7.10 (s, 1H), 6.56 (br s, —NH₂).

Yield: 7% over 2 steps.

### Example 43

### N-[2-Amino-6-(quinolin-5-yloxy)-pyrimidin-4-yl]-3-chloro-benzamide

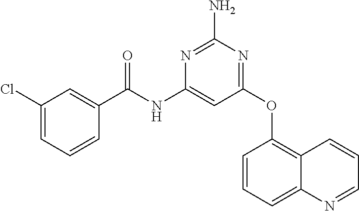 [SMILES: Nc1nc(NC(=O)c2cccc(Cl)c2)cc(Oc2cccc3ncccc23)n1]

ES-MS (M+H)⁺ m/z 394.0.

¹H NMR (400 MHz, DMSO-d₆): δ 10.87 (s, 1H), 8.97 (d, J=2.4 Hz, 1H), 8.25 (d, J=8.4 Hz, 1H), 7.96-8.01 (m, 3H), 7.90 (d, J=7.6 Hz, 1H), 7.82 (t, J=8.0 Hz, 1H), 7.67 (d, J=7.6 Hz, 1H), 7.47-7.59 (m 4H), 7.09 (s, 1H), 6.48 (br s, —NH₂).

Yield: 6% over 2 steps.

### Example 44

### N-[2-Amino-6-(pyridin-4-yloxy)-pyrimidin-4-yl]-3-chloro-benzamide

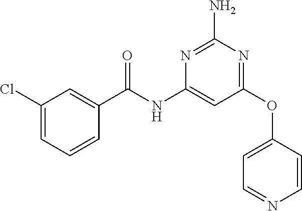 [SMILES: Nc1nc(NC(=O)c2cccc(Cl)c2)cc(Oc2ccncc2)n1]

ES-MS (M+H)⁺ m/z 342.0.

¹H NMR (400 MHz, DMSO-d₆): δ 11.07 (s, 1H), 8.39 (d, J=8.0 Hz, 2H), 8.04 (s, 1H), 7.94 (d, J=7.6 Hz, 1H), 7.69 (d, J=7.2 Hz, 1H), 7.54-7.60 (m, 2H), 6.93 (br s, —NH₂), 6.29 (d, J=8.0 Hz, 2H).

Yield: 10% over 2 steps.

### Example 45

### N-[2-Amino-6-(tetrahydro-pyran-4-yloxy)-pyrimidin-4-yl]-3-chloro-benzamide

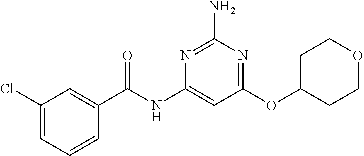 [SMILES: Nc1nc(NC(=O)c2cccc(Cl)c2)cc(OC2CCOCC2)n1]

ES-MS (M+H)⁺ m/z 348.9.

¹H NMR (400 MHz, DMSO-d₆): δ 10.59 (s, 1H), 7.95 (d, J=2.0 Hz, 1H), 7.86 (d, J=8.0 Hz, 1H), 7.67 (d, J=8.0 Hz, 1H), 7.49 (t, J=8.0 Hz, 1H), 6.73 (s, 1H), 6.41 (br s, —NH₂), 5.14 (m, 1H), 3.82 (m, 2H), 3.40-3.46 (m, 2H), 1.94 (m, 2H), 1.58 (m, 2H).

Yield: 2.8% over 2 steps.

### Example 46

### N-[2-Amino-6-(4-amino-cyclohexyloxy)-pyrimidin-4-yl]-3-chloro-benzamide; HCl salt

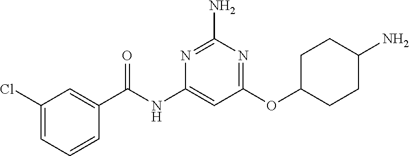 [SMILES: Nc1nc(NC(=O)c2cccc(Cl)c2)cc(OC2CCC(N)CC2)n1]

ES-MS (M+H)⁺ m/z 361.9.

¹H NMR (400 MHz, DMSO-d₆): δ 10.87 (s, 1H), 7.91-8.01 (m, 3H), 7.68 (d, J=9.2 Hz, 1H), 7.54 (t, J=8.0 Hz, 1H), 6.69 (s, 1H), 4.93 (m, 1H), 3.6-3.71 (m, 3H), 3.07 (m, 2H), 2.01-2.10 (m, 2H), 1.52 (m, 2H).

Yield: 1.4% over 3 steps including the final step involving BOC-deprotection performed in standard conditions.

### Example 47

### N-[2-Amino-6-(1-methyl-piperidin-4-yloxy)-pyrimidin-4-yl]-3-chloro-benzamide

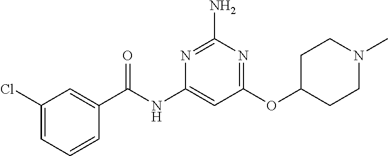 [SMILES: CN1CCC(Oc2cc(NC(=O)c3cccc(Cl)c3)nc(N)n2)CC1]

ES-MS (M+H)⁺ m/z 361.9.

¹H NMR (400 MHz, DMSO-d₆): δ 10.60 (s, 1H), 7.98 (s, 1H), 7.89 (d, J=7.6 Hz, 1H), 7.65 (d, J=8.0 Hz, 1H), 7.52 (dd, J=7.6, 8.0 Hz, 1H), 6.76 (s, 1H), 6.35 (br s, —NH₂), 5.00 (m, 1H), 2.61 (m, 2H), 2.18 (s, 3H), 2.16 (m, 2H), 1.91 (m, 2H), 1.66 (m, 2H).

Yield: 0.7% over 2 steps.

### Example 48

### N-(2-Amino-6-methoxy-pyrimidin-4-yl)-3-chloro-benzamide

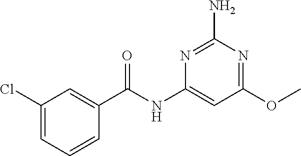 [SMILES: COc1cc(NC(=O)c2cccc(Cl)c2)nc(N)n1]

ES-MS (M+H)⁺ m/z 278.9.

¹H NMR (400 MHz, DMSO-d₆): δ 10.60 (s, 1H), 7.99 (s, 1H), 7.90 (d, J=8.4 Hz, 1H), 7.65 (d, J=8.0 Hz, 1H), 7.52 (dd, J=8.0, 8.4 Hz, 1H), 6.81 (s, 1H), 6.42 (br s, —NH₂), 3.82 (s, 3H).

Yield: 4.45% over 2 steps.

### Example 49

### N-(2-Amino-6-chloro-pyrimidin-4-yl)-3-chloro-benzamide

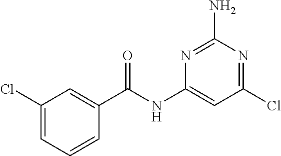 [SMILES: Nc1nc(Cl)cc(NC(=O)c2cccc(Cl)c2)n1]

ES-MS (M+H)⁺ m/z 284.0.

¹H NMR (400 MHz, DMSO-d₆): δ 11.01 (s, 1H), 8.01 (s, 1H), 7.90 (d, J=8.0 Hz, 1H), 7.68 (d, J=8.4 Hz, 1H), 7.54 (dd, J=7.6, 8.0 Hz, 1H), 7.39 (s, 1H), 6.93 (br s, —NH₂).

Yield: 3.8% yield.

### Example 50

### N-[2-Amino-6-(2-methylamino-ethoxy)-pyrimidin-4-yl]-3-chloro-benzamide

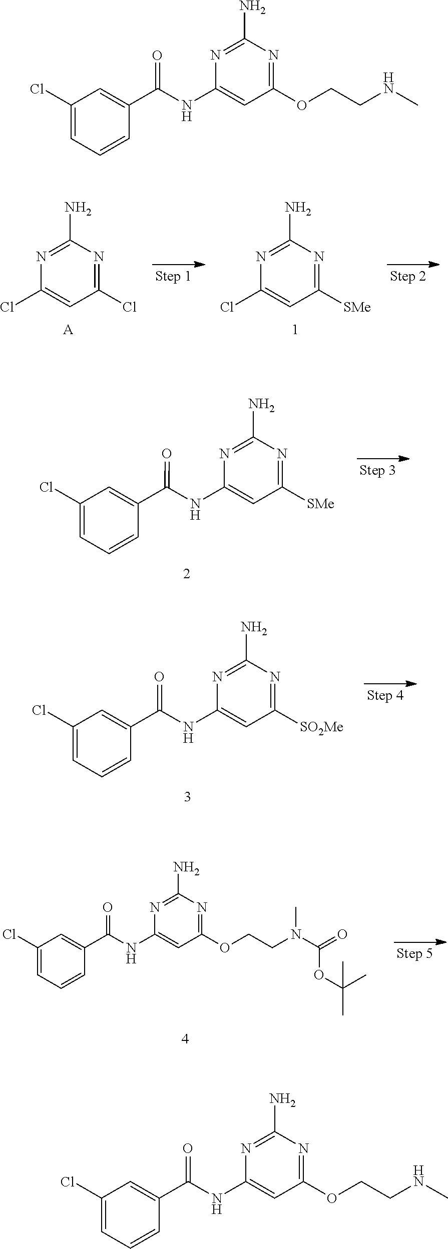

Sodium methyl sulfide (1.1 eq) was added to a solution of 4,6-dichloropyrimidin-2-amine (1.0 eq) in dimethylformamide (5 vol) and the reaction mixture was heated to 80° C. for 3 h. After consumption of starting materials, reaction medium was quenched into ice-cold water to obtain a white solid, which was filtered; washed with water and vacuum dried to afford compound 4-chloro-6-(methylthio)pyrimidin-2-amine in 70% yield.

A mixture of 3-chlorobenzamide (0.7 eq), 4-chloro-6-(methylthio)pyrimidin-2-amine (1 eq), Pd₂(dba)₃ (0.1 eq), XantPhos (0.2 eq) and sodium tertbutoxide (0.7 eq) in dioxane (2 mL) was heated at 140° C. for 0.5 h under nitrogen atmosphere. The mixture was filtered through Celite and the filtrate was concentrated to obtain a residue which was purified by flash column chromatography (dichloromethane/methanol) to afford desired product N-(2-amino-6-(methylthio)pyrimidin-4-yl)-3-chlorobenzamide in 15% yield.

A solution of Oxone (6.0 eq) in water (20 mL) was added to a stirred solution of N-(2-amino-6-(methylthio)pyrimidin-4-yl)-3-chlorobenzamide (1.0 eq) in acetone (20 mL) and the whole mixture was stirred at room temperature for 16 h. After consumption of starting materials, acetone was concentrated and the residue was diluted with water (200 mL); extracted in dichloromethane (2×50 mL). The combined organic phases were washed with brine solution (50 mL) followed by water (50 mL) and the resulting organic layer was dried over anhydrous sodium sulphate and concentrated. The crude product was washed with diethyl ether followed by n-pentane to afford the desired product N-(2-amino-6-(methylsulfonyl)pyrimidin-4-yl)-3-chlorobenzamide in 40% yield.

N-(2-amino-6-(methylsulfonyl)pyrimidin-4-yl)-3-chlorobenzamide (1 eq) was added to a cooled suspension of the corresponding hydroxyl compound (2 eq), and sodium hydride (2.5 eq) in tetrahydrofuran (10 vol) and irradiated in microwave at 900 W for 10 minutes. The reaction mixture was cooled to room temperature, diluted with water (20 mL) and extracted with ethyl acetate (2×20 mL). The combined organic extract was washed with brine solution (10 mL) followed by water (20 mL) and the resulting organic layer was dried over anhydrous sodium sulphate and concentrated. The crude product, tert-butyl (2-((2-amino-6-(3-chlorobenzamido)pyrimidin-4-yl)oxy)ethyl)(methyl)carbamate, was taken to the next step without further purification.

Trifluoroacetic acid was added to a dichloromethane solution of tert-butyl (2-((2-amino-6-(3-chlorobenzamido)pyrimidin-4-yl)oxy)ethyl)(methyl)carbamate at 0° C. and the whole reaction mixture was stirred at room temperature for 2 h. After complete consumption of starting materials, reaction mixture was quenched in water; basified using sat. sodium bicarbonate solution and extracted in ethyl acetate (2×20 mL). The combined organic extract was washed with brine solution (10 mL) followed by water (30 mL) and the resulting organic layer was dried over anhydrous sodium sulphate and concentrated. The residue was washed with diethyl ether to afford pure product N-(2-amino-6-(2-(methylamino)ethoxy)pyrimidin-4-yl)-3-chlorobenzamide in a 0.5% yield over 5 steps.

ES-MS (M+H)⁺ m/z 321.9.

¹H NMR (400 MHz, DMSO-d₆): δ 10.62 (s, 1H), 7.99 (s, 1H), 7.90 (d, J=7.6 Hz, 1H), 7.66 (d, J=8.0 Hz, 1H), 7.52 (dd, J=7.6, 8.0 Hz, 1H), 6.81 (s, 1H), 6.42 (br s, —NH₂), 4.26 (t, J=5.6 Hz, 2H), 2.80 (t, J=5.6 Hz, 2H), 2.31 (s, 3H).

### General Procedure X

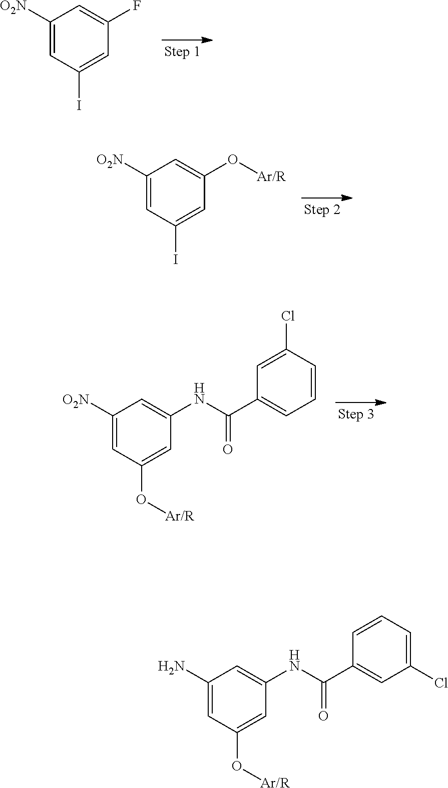

1-Fluoro-3-iodo-5-nitrobenzene (1 eq) was added to a cooled suspension of the appropriate hydroxyl compound (1.2 eq) (ex: 3-chlorophenol), cesium carbonate (1.5 eq) in dimethylformamide (5 mL) and heated at 900 W for 5 minutes (if irradiated in microwave); or heated to 130° C. for 2 h (if heated conventionally). The reaction mixture was cooled to room temperature diluted with water (10 mL) and extracted in ethyl acetate (2×). The combined organic extract was washed with brine solution and the resulting organic layer was separated; dried over anhydrous sodium sulphate and concentrated. The crude was either taken further to next step without any purification or purified by flash column chromatography (ethyl acetate/Hexanes) to yield the desired arylether intermediate (ex: 1-(3-chlorophenoxy)-3-iodo-5-nitrobenzene).

A mixture of 3-chlorobenzamide (1.5 eq), the appropriate arylether (1 eq) (ex: 1-(3-chlorophenoxy)-3-iodo-5-nitrobenzene), Pd₂(dba)₃ (0.1 eq)/Pd(OAc)₂ (0.05 eq), XantPhos (0.2 eq) and cesium carbonate (2 eq)/sodium tertbutoxide (1.5 eq) in dioxane:toluene (1:100) was heated at 110° C. for 4 h under nitrogen atmosphere. The mixture was filtered through celite and the filtrate was concentrated to obtain a residue which was purified by flash column chromatography (dichloromethane/methanol) to afford the desired amide intermediate (ex: 3-chloro-N-(3-(3-chlorophenoxy)-5-nitrophenyl)benzamide).

NH₄Cl (6 eq) was added portion-wise to a suspension of the appropriate nitro intermediate (ex: 3-chloro-N-(3-(3-chlorophenoxy)-5-nitrophenyl)benzamide) (1 eq) and Fe (2 eq) in EtOH:water (3:1) at room temperature and the reaction mixture was refluxed at 100° C. After 1 h, the reaction mass was filtered through celite and concentrated. The residue was dissolved in water, basified to pH˜8 and extracted with ethyl acetate (2×). Combined organic extracts were dried over anhydrous sodium sulphate and the obtained residue was either purified by flash column chromatography (ethyl acetate/Hexanes) or preparative HPLC to afford the desired aniline product (ex: N-[3-amino-5-(3-chloro-phenoxy)-phenyl]-3-chloro-benzamide).

### Example 51

### N-[3-Amino-5-(3-chloro-phenoxy)-phenyl]-3-chloro-benzamide

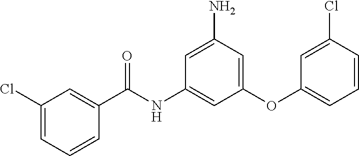 [SMILES: Nc1cc(NC(=O)c2cccc(Cl)c2)cc(Oc2cccc(Cl)c2)c1]

ES-MS (M+H)⁺ m/z 374.0.

¹H NMR (400 MHz, DMSO-d₆): δ 10.12 (s, 1H), 7.94 (s, 1H), 7.85 (d, J=7.2 Hz, 1H), 7.64 (d, J=8.0 Hz, 1H), 7.54 (dd, J=7.6, 8.0 Hz, 1H), 7.41 (d, J=8.4 Hz, 1H), 7.17 (d, J=8.0 Hz, 1H), 7.06 (s, 1H), 6.96-7.00 (m, 2H), 6.64 (s, 1H), 6.01 (s, 1H), 5.40 (br s, —NH₂).

Yield: 36% over 3 steps.

### Example 52

N-[3-Amino-5-(quinolin-3-yloxy)-phenyl]-3-chloro-benzamide.

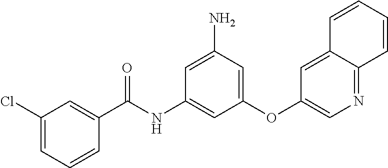 [SMILES: Nc1cc(NC(=O)c2cccc(Cl)c2)cc(Oc2cnc3ccccc3c2)c1]

ES-MS (M+H)⁺ m/z 390.0.

¹H NMR (400 MHz, DMSO-d₆): δ 10.12 (s, 1H), 8.78 (s, 1H), 8.03 (d, J=8.4 Hz, 1H), 7.83-7.97 (m, 4H), 7.51-7.71 (m, 4H), 7.00 (s, 1H), 6.67 (s, 1H), 6.08 (s, 1H), 5.41 (br s, —NH₂).

Yield: 3.5% over 3 steps.

### Example 53

### N-[3-Amino-5-(3-cyano-phenoxy)-phenyl]-3-chloro-benzamide)

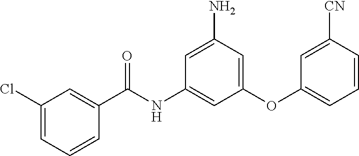 [SMILES: N#Cc1cccc(Oc2cc(N)cc(NC(=O)c3cccc(Cl)c3)c2)c1]

ES-MS (M+H)⁺ m/z 364.0.

¹H NMR (400 MHz, DMSO-d₆): δ 10.11 (s, 1H), 7.94 (s, 1H), 7.85 (d, J=7.2 Hz, 1H), 7.64 (d, J=8.0 Hz, 1H), 7.57-7.58 (m, 2H), 7.53 (s, 1H), 7.51 (s, 1H), 7.48 (s, 1H), 7.34-7.37 (m, 1H), 6.96 (s, 1H), 6.63 (s, 1H), 5.40 (br s, —NH₂).

Yield: 2.5% over 3 steps.

### Example 54

### N-[3-Amino-5-(4-cyano-phenoxy)-phenyl]-3-chloro-benzamide

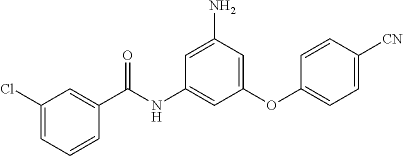 [SMILES: N#Cc1ccc(Oc2cc(N)cc(NC(=O)c3cccc(Cl)c3)c2)cc1]

ES-MS (M+H)⁺ m/z 364.0

¹H NMR (400 MHz, DMSO-d₆): δ 10.14 (s, 1H), 7.95 (s, 1H), 7.84-7.87 (m, 3H), 7.64 (d, J=8.4 Hz, 1H), 7.55 (dd, J=7.6, 8.0 Hz, 1H), 7.14 (d, J=7.0 Hz, 1H), 6.99 (t, J=2.0 Hz, 1H), 6.71 (t, J=2.0 Hz, 1H), 6.07 (t, J=2.0 Hz, 1H), 5.44 (br s, —NH₂).

Yield: 1.9% over 3 steps.

The following compounds, which are according to the invention and have been tested according to the protocols described herein, have been obtained from commercial providers as indicated below:

### Example 55

N-(3-aminophenyl)-3-chlorobenzamide purchased from Prince-princeton biomolecular researchton (Ref OSKK_339340)

### Example 56

N-(3-aminophenyl)-3-iodobenzamide purchased from Prince-princeton biomolecular researchton (Ref OSSK_976596)

### Example 57

N-(3-aminophenyl)-3-(trifluoromethyl)benzamide purchased from Enamine (Ref Z285189920)

### Example 58

N-(3-amino-5-(pyridin-3-yloxy)phenyl)-3-chlorobenzamide purchased from Specs (Ref AK-968/40162671)

### Example 59

N-(3-aminophenyl)-3-methylbenzamide purchased from Prince-princeton biomolecular researchton (Ref OSSK_980622)

### Example 60

N-(4-amino-3-methoxyphenyl)-3-chlorobenzamide purchased from Enamine (Ref BBV-32589144)

### Example 61

N-(3-amino-4-methoxyphenyl)-3-chlorobenzamide purchased from Enamine (Ref BBV-25483249)

### Biological Assays

Compounds according to the present invention are capable of binding alosterically to mutated β-galactosidase enzyme thereby stabilizing the enzyme against denaturation and enhancing its catalytic activity.

### Material and Methods

### Differential Scanning Fluorimetry (DSF).

The capacity of the compounds of the invention to stabilize β-galactosidase was assessed by differential scanning fluorimetry technique. The thermal denaturation of purified human native enzyme was monitored in the presence of the extrinsic fluorescent probe SYPRO Orange (Sigma-Aldrich, St. Louis, Mo.). Compounds were dissolved in 100% DMSO and diluted into the protein buffer to achieve final concentrations of 2% DMSO.

β-galactosidase pure protein (Novoprotein, NJ) 10 microl of 2.5 μM in 20 mM Tris pH 7.5, 150 mM NaCl (final concentration 1 μM) and 12.5 μl of the different compound solutions were dispensed into 96-well PCR-plates (LightCycler480 Multiwell Plate 96, Roche Diagnostics) and incubated 10 min on ice. Then 2.5 μl 50×SYPRO Orange was added until a final volume of 25 μl sealing the plate with aluminium foil, vortex and centrifuge for 2 min.

Plates were loaded into a LightCycler 480 System II (Roche Applied Science, Indianapolis) for thermal denaturation. The increase in SYPRO Orange fluorescence intensity associated with protein unfolding (λexcitation=465 nm, λemission=580 nm) was monitored as a measure of thermal denaturation. Unfolding curves were recorded from 20 to 95° C., at a scan rate of 2° C./min. The experimental unfolding curves were smoothed, normalized, and analyzed using in-house software. The melting temperature (Tm) was calculated as the temperature at which half the protein is in the unfolded state.

The biological activity (capacity to stabilize β-Galactosidase A against denaturation) of the compounds in the differential scanning fluorimetry (DSF) was normalized by the activity shown by 1-deoxygalactonojirimycin in the same assay.

(1-deoxygalactonojirimycin or DGJ, also known as migalastat or Amigal™ is a compound acting as pharmacological chaperone of β-Galactosidase A, now in Phase III for Fabry disease and also act as pharmacological chaperone for acid β-galactosidase)

The capacity to stabilize β-Galactosidase against denaturation is denoted as follows:
ΔTm GLB1 in the presence of the tested compound/ΔTm GLB1 in the presence of migalastat >1 is shown as A
ΔTm GLB1 in the presence of the tested compound/ΔTm GLB1 in the presence of migalastat between 0.5 and 1 is shown as B
ΔTm GLB1 in the presence of the tested compound/ΔTm GLB1 in the presence of migalastat between 0.1 and 0.5 is shown as C

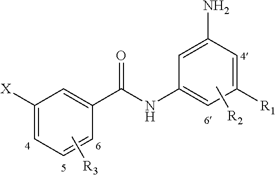

| Example | X | R₁ | R₂ | R₃ | Activity |
|---|---|---|---|---|---|
| 9 | Cl | H | 6′-F | H | C |
| 10 | Cl | H | 4′-Met | H | C |
| 11 | Cl | H | 4′-Met; 6′-F | H | B |
| 12 | Br | H | H | H | A |
| 13 | Cl | H | H | 5-Cl | B |
| 14 | Cl | H | H | 6-CF₃ | C |
| 15 | Cl | H | H | 6-Cl | C |
| 17 | Cl | H | H | 6-OMe | C |
| 19 | Cl | H | H | 2-Me | C |
| 20 | Cl | H | H | 4-OMe | A |
| 21 | Cl | H | H | 5-OMe | C |
| 55 | Cl | H | H | H | A |
| 56 | I | H | H | H | B |
| 57 | CF₃ | H | H | H | B |
| 58 | Cl | —O-3-Py | H | H | A |
| 59 | Me | H | H | H | C |

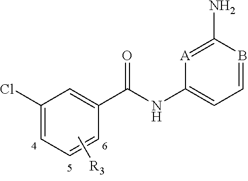

| Example | A | B | R₃ | Activity |
|---|---|---|---|---|
| 2 | N | N | 4-OMe | B |
| 3 | N | N | 4-F | C |
| 7 | N | C | H | B |
| 8 | N | N | H | A |
| 34 | C | N | H | C |

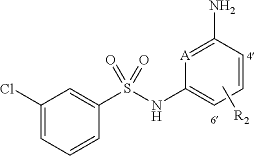

| Example | A | R₂ | Activity |
|---|---|---|---|
| 22 | C | 6′-F | B |
| 23 | N | 4′-Me, 6′-F | C |
| 24 | C | H | C |

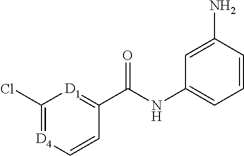

| Example | D₁ | D₄ | Activity |
|---|---|---|---|
| 25 | C | N | C |
| 26 | N | C | C |

Enhancement of β-Galactosidase Activity Measured in Transfected Cells with WT or Mutant Proteins p.T420K, p.R457Q, p.Y83C and R201H

### Vector Construction

The coding region of Human wild-type β-galactosidase cDNA was amplified by PCR in two fragments that were ligated and cloned in a pUC18 vector. Mutations p.T420K, p.R457Q, p.Y83C and p.R201H were generated by site-directed mutagenesis using the QuickChange™ Site-Directed Mutagenesis XL kit (Stratagene, La Jolla, Calif.) according to the manufacturer's instructions. The constructs were resequenced to ensure that no spurious mutation had been introduced. For protein expression, the wild-type and mutated cDNAs were subcloned into the pcDNA3.1 expression vector.

### Cell Culture and Transfection

COS-7 cells were cultured in 100 mm diameter tissue culture dishes with DMEM (Sigma-Aldrich, St. Louis, Mo.), 10% fetal bovine serum (Life Technologies S.A., Carlsbad, Calif.), and antibiotics. For transfection with wild-type and mutant β-galactosidase cDNAs, 8×10⁴ cells per well were plated in 12-well microplates. Twenty-four hours later, 1.6 μg of the corresponding plasmid mixed with 2.5 μl of Lipofectamine™ 2000 Reagent (Life Technologies S.A., Carlsbad, Calif.) was added to each well. As a negative control, a pcDNA vector carrying antisense β-galactosidase cDNA was transfected. After 6 hours of incubation at 37° C., transfection medium was removed, and the cells were exposed to fresh medium adding the compounds at the desired concentration. After 48 hours of incubation, the cells were washed with PBS and incubated with or without the compounds for another 24 h. At the end of this period, the medium was removed, and cells were washed and exposed for 4 hours to complete medium. Then, cells were collected and centrifuged at 13000 rpm for 5 minutes. Cellular pellets were washed twice with PBS and stored at −80° C. until the enzymatic analysis was performed.

### Enzyme Activity

β-galactosidase activity in cell lysates was measured by using 4-MU-β-galactopyranoside substrate (Sigma-Aldrich, St. Louis, Mo.). Lysates were resuspended in 200 μl of 0.9% NaCl containing 0.01% triton X-100 lysis buffer to promote membrane disruption. The cell suspension was sonicated and centrifuged at 13000 rpm 2 min to remove insoluble materials. Then, lysates were mixed with 4-MU-β-galactopyranoside in 100 mM citrate buffer (pH=4) 100 mM NaCl for 30 min at 37° C. The reaction was terminated by adding 200 mM glycin-NaOH buffer (pH=10.7). The liberated 4-MU was measured with a fluorescence reader (excitation 340 nm, emission 460 nm, Modulus Microplate Multimode Reader, Turner Biosystems). Protein quantification was determined using BCA protein assay kit (Pierce BCA Protein Assay Kit, Thermo Fisher Scientific Inc., Waltham, Mass.).

Measurements were interpolated in a 4-MU standard curve and normalized by protein quantity. Enzyme activities were expressed in treated cells as X-fold increase in comparison with non-treated cells (X=1 represents no enhancement).

In COS-7 cells transiently transfected with human GLB1 mutants p.T420K, p.R457Q, p.Y83C and p.R201H, compounds of the invention tested at 12.5-100 μM significantly enhance the enzymatic activity with a value equal or greater than 20%, when compared with the residual activity of non-treated cells. When compared with activity of cells transfected with wilde-type GLB1, such enhancement of activity is reported in literature as a meaningful increase on enzymatic activity. Mutants p.T420K and p.R457Q are responsible for adult type III GM1-gangliosidosis, and mutant p.Y83C causes Morquio B disease. Mutation p.R201H has been found in patients with juvenile (type II) and adult (type III) GM1-gangliosidosis and Morquio B disease.

1. A compound of general formula (I), 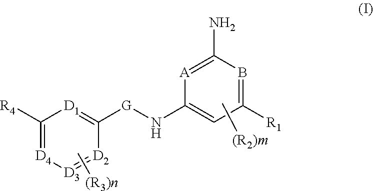
wherein:
A or B are independently selected from nitrogen and —CH═;
D₁, D₂, D₃ and D₄ are nitrogen or ═CH— with the proviso that 0, 1 or 2 of D₁, D₂, D₃ and D₄ are nitrogen while the rest are —CH═;
G is selected from —C(═O)— and —SO₂—;
R₄ is selected from halogen, methyl, —CF₃ and —OCF₃;
R₁ is selected from hydrogen, halogen, hydroxy, —CN, —ORa, —SRa, —N(Rb)₂, —C₁₋₄ alkyl, —C(═O)ORc, —C(═O)N(Rb)₂, —SO₂N(Rb)₂, —C₃₋₆ cycloalkyl and —C₃₋₇ heterocyclyl; said alkyl, cycloalkyl and heterocyclyl groups are optionally substituted with 1, 2 or 3 groups independently selected from halogen, hydroxy, —C₁₋₄ alkyl, —N(Rb)₂, methoxy, -haloC₁₋₄alkyl, -dihaloC₁₋₄alkyl, -trihaloC₁₋₄alkyl, halomethoxy, dihalomethoxy, and trihalomethoxy;
each Ra or Rb independently represents, hydrogen, —C(═O)Rd, —SO₂Rd, —C₁₋₄ alkyl, —C₃₋₆ cycloalkyl, —C₅₋₁₀ aryl, —C₅₋₁₀ heteroaryl, or —C₃₋₇ heterocyclyl; said alkyl, cycloalkyl, aryl, heteroaryl or heterocyclyl groups optionally being substituted with 1, 2 or 3 groups independently selected from halogen, hydroxy, amine, —C₁₋₄ alkyl, —CN, methoxy, aryl, substituted heteroaryl, -haloC₁₋₄alkyl, -dihaloC₁₋₄alkyl, -trihaloC₁₋₄alkyl, halomethoxy, dihalomethoxy and trihalomethoxy;
each Rd or Rc independently represent hydrogen, —C₁₋₄ alkyl, —C₃₋₆ cycloalkyl, —C₅₋₁₀ aryl, —C₅₋₁₀ heteroaryl, or —C₃₋₇ heterocyclyl; said alkyl, cycloalkyl, aryl, heteroaryl or heterocyclyl groups optionally being substituted with 1, 2 or 3 groups independently selected from halogen, hydroxy, methoxy, halomethoxy, dihalomethoxy, and trihalomethoxy;
each R₃ is independently selected from hydrogen, halogen, hydroxy, —CN, —ORa′, —SRa′, —N(Rb′)₂ and —C₁₋₄ alkyl; said —C₁₋₄ alkyl group optionally being substituted with 1, 2 or 3 groups independently selected from halogen, hydroxy and —N(Rb)₂;
each Ra′ or Rb′ independently represent, hydrogen, —C(═O)Rd, —SO₂Rd, —C₁₋₄ alkyl, —C₃₋₆ cycloalkyl, —C₅₋₁₀ aryl, —C₅₋₁₀ heteroaryl, or —C₃₋₇ heterocyclyl; said alkyl, cycloalkyl, aryl, heteroaryl or heterocyclyl groups optionally being substituted with 1, 2 or 3 groups independently selected from halogen, hydroxy, amine, methoxy, substituted aryl, substituted heteroaryl, halomethoxy, dihalomethoxy and trihalomethoxy;
each R₂ is independently selected from hydrogen, halogen, hydroxy, —CN, —ORa, —SRa, —N(Rb)₂, —C₁₋₄ alkyl, —C₃₋₆ cycloalkyl, —C₅₋₁₀ aryl and —C₃₋₇ heterocyclyl; said alkyl, cycloalkyl, aryl or heterocyclyl groups optionally being substituted with 1, 2 or 3 groups independently selected from halogen, hydroxy, —C₁₋₄ alkyl, —N(Rb)₂, methoxy, -haloC₁₋₄alkyl, -dihaloC₁₋₄alkyl, -trihaloC₁₋₄alkyl, halomethoxy, dihalomethoxy, and trihalomethoxy;
n and m have independently a value selected from 0, 1 and 2;
or a solvate or a salt thereof for use in the prevention or treatment of a condition associated with the alteration of the activity of GLB1.

2. A compound for use according to claim 1 wherein G is a group —C(═O)—.

3. A compound for use according to anyone of claims 1 and 2 wherein R₄ is selected from chloro, bromo and —CF₃.

4. A compound for use according to anyone of claims 1 to 3 wherein R₁ is selected from hydrogen, halogen, —ORa and —C₁₋₄ alkyl optionally substituted with 1, 2 or 3 groups independently selected from halogen, hydroxy, —C₁₋₄ alkyl, —N(Rb)₂, methoxy, -haloC₁₋₄alkyl, -dihaloC₁₋₄alkyl, -trihaloC₁₋₄alkyl, halomethoxy, dihalomethoxy, and trihalomethoxy.

5. A compound for use according to anyone of claims 1 to 4 wherein Ra and Rb are independently selected from —C₁₋₄ alkyl, —C₃₋₆ cycloalkyl, —C₅₋₁₀ aryl, —C₅₋₁₀ heteroaryl, and —C₃₋₇ heterocyclyl; said alkyl, cycloalkyl, aryl, heteroaryl, or heterocyclyl groups optionally being substituted with 1, 2 or 3 groups independently selected from halogen, hydroxy, amine, —C₁₋₄ alkyl, —CN, methoxy, substituted aryl, substituted heteroaryl, -haloC₁₋₄alkyl, -dihaloC₁₋₄alkyl, -trihaloC₁₋₄alkyl, halomethoxy, dihalomethoxy and trihalomethoxy.

6. A compound for use according to anyone of claims 1 to 5 wherein Rc and Rd are independently selected from —C₁₋₄ alkyl, —C₅₋₁₀ aryl, and —C₃₋₇ heterocyclyl; said alkyl, cycloalkyl, aryl or heterocyclyl groups optionally being substituted with 1, 2 or 3 groups independently selected from halogen, hydroxy, methoxy, halomethoxy, dihalomethoxy, and trihalomethoxy.

7. A compound for use according to anyone of claims 1 to 6 wherein R₃ is selected from hydrogen, halogen, —ORa′ and —C₁₋₄ alkyl.

8. A compound for use according to anyone of claims 1 to 7 wherein Ra′ and Rb′ are independently selected from —C₁₋₄ alkyl, —C₃₋₆ cycloalkyl, —C₅₋₁₀ aryl, and —CO₃₇ heterocyclyl; said alkyl, cycloalkyl, aryl or heterocyclyl groups optionally being substituted with 1, 2 or 3 groups independently selected from halogen, hydroxy, amine, methoxy, substituted aryl, substituted heteroaryl, halomethoxy, dihalomethoxy and trihalomethoxy.

9. A compound for use according to anyone of claims 1 to 8 wherein R₂ is independently selected from hydrogen, halogen and —C₁₋₄ alkyl.

10. A compound for use according to anyone of claims 1 to 9 wherein m represents 0 or 1.

11. A compound for use according to anyone of claims 1 to 10 wherein n represents 0 or 1.

12. A compound for use according to anyone of claims 1 to 11 wherein the condition associated with the alteration of the activity of GLB1 is selected from the group consisting of GM1 gangliosidoses and Morquio syndrome, type B.

13. Use of a compound of formula (I) as defined in any one of claims 1 to 11 or a salt or solvate thereof, in the preparation of a medicament for the prevention or treatment of a condition associated with the alteration of the activity of GLB1.

14. Use according to claim 13, wherein the condition associated with the alteration of the activity of GLB1 is selected from the group consisting of GM1 gangliosidoses and Morquio syndrome, type B.

15. A method for the prevention or treatment of a condition associated with the alteration of the activity of GLB1, which comprises the administration to a patient needing such prevention or treatment, of a therapeutically effective amount of at least one compound of formula (I) as defined in any one of claims 1 to 11 or a salt or solvate thereof.

16. The method according to claim 15, wherein the condition associated with the alteration of the activity of GLB1 is selected from the group consisting of GM1 gangliosidoses and Morquio syndrome, type B.

17. A pharmaceutical composition comprising a compound as defined in anyone of claims 1 to 11.

18. A compound of formula (Ia) 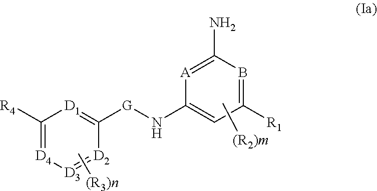
wherein:
A or B are independently selected from nitrogen and —CH═ with the proviso that at least one of A and B is a nitrogen atom;
D₁, D₂, D₃ and D₄ are nitrogen or ═CH— with the proviso that 0, 1 or 2 of D₁, D₂, D₃ and D₄ are nitrogen while the rest are —CH═;
G is selected from —C(═O)— and —SO₂—;
R₄ is an halogen atom;
R₁ is selected from hydrogen, halogen, hydroxy, —CN, —ORa, —SRa, —N(Rb)₂, —C₁₋₄ alkyl, —C(═O)ORc, —C(═O)N(Rb)₂, —SO₂N(Rb)₂, —C₃₋₆ cycloalkyl and —C₃₋₇ heterocyclyl; said alkyl, cycloalkyl and heterocyclyl groups are optionally substituted with 1, 2 or 3 groups independently selected from halogen, hydroxy, —C₁₋₄ alkyl, —N(Rb)₂, methoxy, -haloC₁₋₄alkyl, -dihaloC₁₋₄alkyl, -trihaloC₁₋₄alkyl, halomethoxy, dihalomethoxy, and trihalomethoxy;
each Ra or Rb independently represents, hydrogen, —C(═O)Rd, —SO₂Rd, —C₁₋₄ alkyl, —C₃₋₆ cycloalkyl, —C₅₋₁₀ aryl, —C₅₋₁₀ heteroaryl, or —C₃₋₇ heterocyclyl; said alkyl, cycloalkyl, aryl, heteroayl or heterocyclyl groups optionally being substituted with 1, 2 or 3 groups independently selected from halogen, hydroxy, amine, —C₁₋₄ alkyl, —CN, methoxy, aryl, substituted heteroaryl, -haloC₁₋₄alkyl, -dihaloC₁₋₄alkyl, -trihaloC₁₋₄alkyl, halomethoxy, dihalomethoxy and trihalomethoxy;
each Rd or Rc independently represent hydrogen, —C₁₋₄ alkyl, —C₃₋₆ cycloalkyl, —C₅₋₁₀ aryl, —C₅₋₁₀ heteroaryl, or —C₃₋₇ heterocyclyl; said alkyl, cycloalkyl, aryl, heteroaryl or heterocyclyl groups optionally being substituted with 1, 2 or 3 groups independently selected from halogen, hydroxy, methoxy, halomethoxy, dihalomethoxy, and trihalomethoxy;
each R₃ is independently selected from hydrogen, halogen, hydroxy, —CN, —ORa′, —SRa′, —N(Rb′)₂ and —C₁₋₄ alkyl; said —C₁₋₄ alkyl group optionally being substituted with 1, 2 or 3 groups independently selected from halogen, hydroxy and —N(Rb)₂;
each Ra′ or Rb′ independently represent, on each occasion when used herein, hydrogen, —C(═O)Rd, —SO₂Rd, —C₁₋₄ alkyl, —C₃₋₆ cycloalkyl, —C₅₋₁₀ aryl, —C₅₋₁₀ heteroaryl, or —C₃₋₇ heterocyclyl; said alkyl, cycloalkyl, aryl, heteroaryl or heterocyclyl groups optionally being substituted with 1, 2 or 3 groups independently selected from halogen, hydroxy, amine, methoxy, substituted aryl, substituted heteroaryl, halomethoxy, dihalomethoxy and trihalomethoxy;
R₂ is selected from hydrogen and fluor;
n has a value selected from 0, 1 and 2;
m has a value selected from 0 and 1;
or a solvate or a salt thereof.
